(19) United States
(12) Patent Application Publication
(10) Pub. No.: US 2019/0292581 A1
(43) Pub. Date: Sep. 26, 2019

(54) PROXIMITY ASSAYS FOR DETECTING NUCLEIC ACIDS AND PROTEINS IN A SINGLE CELL

(71) Applicant: Fluidigm Corporation, South San Francisco, CA (US)

(72) Inventors: Camila Egidio, South San Francisco, CA (US); Ramesh Ramakrishnan, San Jose, CA (US); David Ruff, South San Francisco, CA (US)

(21) Appl. No.: 16/373,507

(22) Filed: Apr. 2, 2019

Related U.S. Application Data
(63) Continuation of application No. 14/477,721, filed on Sep. 4, 2014, now abandoned.
(60) Provisional application No. 61/873,820, filed on Sep. 4, 2013.
(60) Provisional application No. 61/987,401, filed on May 1, 2014.

(51) Int. Cl. C12Q 1/6804; C12Q 1/6848; C12Q 1/6851

(52) U.S. Cl. CPC C12Q 1/6804; C12Q 1/6851; C12Q 1/6848

(57) ABSTRACT

Methods and reagents for detection and analysis of nucleic acids and proteins using proximity extension assays.

### CROSS-REFERENCE TO RELATED APPLICATIONS

This application is a continuation of and claim priority to U.S. patent application Ser. No. 14/477,721, filed Sep. 4, 2014, which claims priority benefit of U.S. provisional application Nos. 61/873,820, filed Sep. 4, 2013 and 61/987,401, filed May 1, 2014, each of which applications is herein incorporated by reference.

### BACKGROUND

Detection and quantification of protein and nucleic acids from individual cells is desirable, but difficult to achieve because of the minute amount of material present in a single cell. Further, unlike bulk samples, a single cell cannot be divided into portions to separately analyze proteins and nucleic acids. Although single molecule detection techniques or mass spectrometry may provide methods for achieving single cell analysis, such methods are expensive. The Proximity Extension Assay (PEA) has been developed that is sensitive enough to detect picogram quantities of protein (see, e.g., Lundberg et al., Nucl. Acids Res. 2011 August; 39(15):e102; epub 2011 Jun. 6, incorporated by reference herein). In one approach, the PEA employs a pair of antibodies, each having a oligonucleotide attached to it. The oligonucleotides contain regions that complement one another. When the antibodies bind to a target protein, the oligonucleotides are in close enough proximity so that the complementary regions from each oligonucleotide hybridize to one another. The addition of a DNA polymerase results in extension of the hybridized oligonucleotides. The extension products can then be detected or quantified.

The invention relates to proximity extension assays employed to detect proteins, nucleic acids, and protein-protein and protein-nucleic acid complex interactions in a single cell.

### BRIEF DESCRIPTION OF ASPECTS OF THE INVENTION

In various aspects, the invention includes, but is not limited to, the following embodiments:

In one aspect, the invention provides a method of detecting an analyte of interest in a single cell, the method comprising: a) isolating the single cell; b) incubating the single cell in a lysing buffer comprising a detergent present at a concentration below the critical micelle concentration to obtain a cell lysate; c) incubating the cell lysate with two or more proximity extension probes in a binding reaction at an incubation temperature from about 15° C. to about 50° C. for a length of time from about 5 minutes to about 6 hours under conditions where the proximity extension probes bind to the target analyte, if present, in the cell lysate; d) incubating the binding reaction with an extension mix that comprises a polymerase, wherein hybridized oligonucleotide components of the proximity extension probe are extended by the polymerase to produce extension products; and e) detecting the extension products. In some embodiments, the binding reaction is diluted, e.g., in a range of from about 1:2 to about 1:20 or from about 1:4 to about 1:10, before the extension mix is added. In some embodiments, at least one of the proximity extension probes comprises an antibody as the analyte binding component. In some embodiments, each of the proximity extension probes comprises an antibody as the analyte binding component. In some embodiments, the reaction is performed in a droplet, a well, or a chamber or channel of a microfluidic device. In some embodiments, the reaction performed in a droplet. In some embodiments, the incubation time of the binding reaction is less than about 3 hours or less than about 2 hours or less than about 1 hour. In some embodiments, the binding reaction is incubated at a temperature from about 25° C. to about 50° C. or from about 30° C. to about 45° C. In some embodiments, the proximity probes are present in the binding reaction at a concentration ranging from about 10 pM to about 50 pM. In some embodiments, steps b through d are performed concurrently. In some embodiments, steps b through d are performed sequentially. In some embodiments, steps b and c are performed concurrently. In some embodiments, step c is performed prior to step d. In such an embodiment, step c may be performed concurrently with step b, or b and c may be performed sequentially. In some embodiments, the detergent is a non-ionic detergent or Zwitterionic detergent. In some embodiments, the method further comprises a reverse transcription reaction or whole genome amplification reaction that is performed following the extension reaction. In some embodiments, a reverse transcription reaction can be performed concurrently with the extension reaction.

In a further aspect, the invention provides a multiplex protein detection method, the method comprising incubating a test sample with a plurality of probes to detect the presence of one or more proteins of interest; and incubating a positive control sample comprising a lysate from thymic epithelial cells with the multiple probes, where the lysate comprises proteins to which the protein-binding moieties of the probes can bind, and detecting binding of the probes to proteins in the lysate, wherein the presence of binding of the multiple probes to cognate proteins in the lysate is a positive control for the multiplex protein detection assay. In some embodiments, the thymic epithelial cells are human epithelial cells. In some embodiments, the lysate is from a single cell.

In a further aspect, the invention provides a method of controlling for assay conditions for a single cell multiplex proximity extension assay to detect the presence of one or more proteins in a sample of interest, the method comprising isolating a single test cell and isolating a thymic epithelial cell, lysing the isolated test cell and the isolated thymic epithelial cell and performing a multiplex proximity detection assay on the lysate of the test cell and the lysate of the thymic epithelial cell; and detecting a product from the extension of hybridized oligonucleotide components of a proximity probe pair in the lysate from the thymic epithelial cell, thereby providing a positive control for the assay conditions for the single cell multiplex detection assay. In some embodiments, the assay is performed in microfluidic device. In some aspects, the invention additionally provides a kit comprising sets of proximity extension probes, for example sets of proximity extension probes for a multiplex assay to identify two or more proteins of interests in a solution, and a lysate from thymic epithelial cells.

In a further aspect, the invention provides a method of detecting a target analyte of interest, typically a protein, present on the surface of a single cell. In some embodiments, the method comprises a) isolating the single cell; b) incubating the single cell with two or more proximity extension probes in a binding reaction under conditions where the proximity extension probes bind to the target analyte, if present, e.g., at an incubation temperature from about 15° C. to about 50° C. for a length of time from about 5 minutes to about 6 hours; c) incubating the binding reaction with an extension mix that comprises a polymerase, wherein hybridized oligonucleotide components of the proximity extension probe are extended by the polymerase to produce extension products; and e) detecting the extension products. In some embodiments, the method further comprises a step of lysing the cells and detecting the presence of intracellular proteins using a proximity extension assay as described herein.

In another aspect, the invention provides a proximity extension detection probe set for detecting interaction of a protein with a single-stranded nucleic acid, wherein the probe set comprises a first proximity probe that comprises a binding region that binds to the protein and a first oligonucleotide comprising an interacting region; and a second proximity probe that comprises an oligonucleotide that comprises a segment that hybridizes to the single stranded nucleic acid and a segment that comprises an interacting region that is complementary to the interacting region of the first proximity probe, wherein, when the protein is bound to the single-stranded nucleic acid, the interacting region of the first probe hybridizes to the complementary segment of the second probe. The invention additionally provides a method of detecting interaction of a protein with a single-stranded nucleic acid, the method comprising performing a proximity extension reaction using such a probe set. In some embodiments, the reaction is performed on a sample obtained from a single cell.

In a further aspect, the invention provides a method of detecting the presence of an antigen, typically a protein antigen, in a sample from a single cell, the method comprising: lysing a single cell to obtain a cell lysate; incubating the lysate with an antigen-binding moiety, which bind to the antigen of interest, where the antigen-binding moiety is immobilized to a solid phase, under conditions in which the antigen-binding moiety binds to the antigen to form an antigen/antigen-binding moiety complex; washing the solid phase comprising the complex; and detecting the complex using a proximity extension assay. In typical embodiments, the method is performed in a microfluidic device. In some embodiments, the antigen-binding moiety is immobilized to a bead. In some embodiments, the lysate is incubated with a plurality of beads and a plurality of proximity extension probe pairs. In some embodiments, the antigen-binding moiety bound to the solid phase is a component of a proximity extension pair. In some embodiments, the antigen/antigen binding moiety complex is incubated with a pair of proximity probes, each of which comprises an antigen binding moiety that binds to a different epitope on the antigen. In typical embodiments, the antigen-binding moiety is an antibody.

In a further aspect, the invention provides a method of detecting the presence of an antigen, typically a protein antigen, in a sample, the method comprising incubating the sample with a proximity extension probe set that comprises three proximity probes, wherein (i) a first probe comprises (a) a binding region that binds to a first epitope of the antigen and (b) an oligonucleotide that comprises a hybridizing region that is complementary to a hybridizing region of the oligonucleotide of a second proximity probe; (ii) the second proximity probe comprises (a) a binding region that binds to a second epitope on the antigen and (b) an oligonucleotide that comprises a first hybridizing region complementary to the hybridizing region of the first probe and a second hybridizing region complementary to a hybridizing region of the third probe; and (iii) a probe that comprises (a) binding region that binds to a third epitope on the antigen and (b) an oligonucleotide that comprises a hybridizing region complementary to the second hybridizing region of the second proximity probe; and detecting the interactions of the proximity probe set. In some embodiments, the sample is from a single cell. In typical embodiments, one or more of the binding regions is an antibody.

In another aspect, the invention provides a proximity extension probe set comprising: a first proximity probe and a second proximity probe, wherein: the first member of the proximity probe pair comprises a first antibody joined to an oligonucleotide that comprises a primer binding site, a first hybridizing region, a spacer, and a second hybridizing region; and the second member of the proximity probe pair comprises an antibody, a primer binding site, a first hybridizing region that is complementary to the first hybridizing region of the first proximity probe, a spacer, and a second hybridizing region that is complementary to the second hybridizing region of the first proximity probe; and further, wherein the primer binding sites are 16 to 24 nucleotides in length, the first hybridizing regions are 6 to 9 nucleotides in length, the spacers are 8 to 15 nucleotides in length, and the second hybridizing regions are 4 to 6 nucleotides in length. In some embodiments, the invention provides a proximity extension reaction mixture comprising such a proximity extension probe set and methods of analyzing a sample for the presence of an analyte, the method comprising detecting the presence of an analyte using such a probe set.

### BRIEF DESCRIPTION OF THE DRAWINGS

FIG. 1 provides a schematic of an example of a proximity probe pair that can be used to detect a protein interaction with a single-stranded nucleic acid (e.g., RNA). In this illustration of an embodiment of the invention, an antibody-based proximity probe specific for the protein is one member of the proximity probe pair. The other member of the proximity probe pair is a chimeric DNA molecule that comprises a region that is specific to the single-stranded nucleic acid and a region that hybridizes to a complementary region on the antibody-based proximity probe.

FIG. 2 illustrates embodiments of the invention in which three separate antibodies are employed in a proximity extension assay.

FIG. 3 illustrates an embodiment of the invention in which three separate proximity probes are used in a proximity extension assay. In this illustration two-Ab-bound oligonucleotides hybridize to a third oligonucleotide bridge for hybridization for polymerase extension.

FIG. 4A and FIG. 4B. A schematic of an illustrative proximity extension probe pair that employs two sets of complementary sequences (FIG. 4A) and an illustration of binding of such a probe pair to a target (FIG. 4B). In this example, each oligonucleotide is 44-nt in length.

FIG. 5 provides data from an experiment showing a background signal comparison: standard Proseek negative control (01_NC-ctrl) vs 1% NP40 Cell Lysis Buffer (NP40+_NC-ctrl). The lysis buffer background is in general 1-3 Cq's lower for the 6 protein targets tested as compared to the kit's negative control.

FIG. 6 shows data from an experiment comparing background signal levels between the Proseek kit's negative control (OI_NC), 0.1% non-ionic detergent buffers (Tw_NC, Tween-20; NDSB_NC, Triton X-100; NP40_NC, NP40) and 1% of NP40. Lower concentration of non-ionic detergent has the same background Cq levels as the Proseek kit's negative control.

FIG. 7A and FIG. 7B providing date from an experiment evaluating background Cq. The graph on the top FIG. 7A shows data from an experiment evaluating the Cq levels for the Proseek kit's negative control vs 1% of NP40 as negative control using 3 different probe concentrations for the incubation step: 100 (ctrl), 66 and 33 pM. For both the Proseek negative control (OI_NC) and the NP40, 33 pM probe concentration showed the lowest background signal detected. In this experiment, only the lower probe concentrations used to detect EpCAM protein levels succeeded in separating true signal from background down to 16 cells (lower graph, FIG. 7B). These experiments were performed on plates using dilutions of K562 cell solution for the different input amounts.

FIG. 8A and FIG. 8B provide data from an experiment comparing background Cq for in various incubation conditions.

FIG. 9A and FIG. 9B provide data from an experiment evaluating background Cq. The graph on the top (FIG. 9A) shows the background signal detected when using the Proseek PEA protocol and modified protocols in which a dilution of the extension template was used for the extension reaction. In average, there is roughly a 4 Cq unit decrease in background signal when a dilution step is included. The added dilution step allowed the detection of EpCAM protein levels down to 16 cells compared to control (original protocol, bottom graph, FIG. 9B).

FIG. 10A and FIG. 10B provide data from an experiment evaluating background Cq. The graph on the top (FIG. 10A) shows the background subtraction from signal for cell inputs down to 16 cells. In this experiment, the standard protocol along with a protocol using lower probe concentration was tested. For Caspase-3, the background signal levels did not allow clear separation from true protein signal even when the lower probe concentration protocol was tested. The graph on the bottom (FIG. 10B) shows the improvements when various modifications to increase the sensitivity in accordance with the invention are performed (the different curves show the different lysis buffers used; the modified protocol does not include the extension template dilution step). Over 5 C_{q} units difference is seen between noise and protein signal for 12 cell input. These experiments were performed on plates using dilutions of K562 cell solution for the different input amounts and on different experimental days by the same person.

FIG. 11A and FIG. 11B illustrate a method of monitoring lysis of a cell(s).

FIG. 12A and FIG. 12B illustrate the C₁™ Single-Cell Auto Prep System. The C₁™ Single-Cell Auto Prep System is composed of a controller instrument FIG. 12A and integrated fluidic circuits (IFC, B) containing 96 individual capture sites and dedicated nano-chambers for downstream reactions.

FIG. 13A-FIG. 13C illustrate the PEA method. FIG. 13A shows that each target-specific antibody is labeled with A or B oligonucleotides (PEA probes). During the incubation step, the PEA probes bind to the specific protein in the sample, bringing the A and B oligonucleotides closer in proximity. Hybridization of a complementary region within the A and B oligonucleotides takes place, followed by extension and amplification of the reporter oligonucleotide in a subsequent step, in presence of a DNA polymerase. Detection of the reporter oligonucleotide is done by qPCR on the BioMark™ System. Cycle threshold of the amplified reporter oligo reflects target protein abundance during the incubation step. FIG. 13B is a representation of the system of independent chambers and valves connected to the 4.5 nL single-cell capture site in the C₁™ IFC. Each one of the 96 capture sites has its own dedicated system of chambers and valves, allowing all PEA steps to take place in a single run for 96 single cells in parallel. FIG. 13C provides a list of of protein targets for the PEA probe panel contained in the Proseek Multiplex Oncology I^{96×96} kit used. Of the 92 protein targets, 25 (around 30%) are strictly secreted and not expected to generate signal when performing single cell analysis. FIG. 13D shows the single-cell-to-result turnaround time for the system.

14 illustrates exemplary characteristic protein expression signatures identified using the system.

FIG. 15A-15D shows targets detected in specific cell lines (FIG. 15A) CRL-7163, (FIG. 15B) MDA-MB-231, (FIG. 15C) HL60, and (FIG. 15D) K562) across two independent C₁™ PEA experiments. (left bars, experiment 1; right bars, experiment 2)

FIG. 16 shows results from PEA on plate-sorted cells and two independent C₁™ PEA experiments on single HL60 cells.

FIG. 17A-17C shows that flow cytometry and immunofluorescence results are consistent with C₁™ PEA results. FIG. 17A shows C₁™ PEA results for two specific targets were validated on HL60 and K562 cells using orthogonal methods. FIG. 17A provides a diagram showing a heat map of the protein expression results for C₁™ PEA and IF for EpCAM (red indicates high expression). FIG. 17C provides an image of two cells that were captured in the C₁™ IFC chamber.

FIG. 18 provides results from seven targets for six different concentrations of probe in the incubation for single cell C₁™-PEA on K562 cells. The Y-axis shows the average Cₜ values for either live cells (as detected with a Live/Dead stain; blue, lower lines) or empty C₁™ positions (i.e. background; red, upper lines) for each of the example seven targets. The number of either live cells or empty positions used to calculate the average Cₜ is given. The standard error for each data point is also shown.

FIG. 19 provides results showing that conditions of 4° C. for 12-16 hs incubation produced the lowest Cq compared to 37° C. incubation for 1 hr.

FIG. 20A and FIG. 20B provides results from an internal PEA control (oligo-reference) showing that there was a relationship between position on chip and PEA performance (Panel A), which was resolved by switching inlets for the PEA mix (i.e. enzymes and PEA solution; Panel B). For both panels, Ct values are shown on the Y-axis and the position numbers are on the X-axis. On the X-axis, to the left of the backslash is the number for the position on the left side of the chip (bars on left side, blue) and to the right of the backslash is the position number for the right side of the chip (bars on right side, red). The arrow in both panels shows the positions which are most proximal to the reagent entry point into the C₁™ IFC to the most distal point from that entry.

FIG. 21. Panel A shows inlet numbering on a C₁™ chip. Panel B shows an illustrative final configuration of PEA reagents loaded to the C₁™ chip carrier.

FIG. 22 provides depicting an illustrative C₁™-PEA reaction on a chip.

### DETAILED DESCRIPTION

### Definitions and Terminology

The terms “a”, “an”, or “the” are generally intended to mean “one or more” unless otherwise indicated.

Where a range of values is provided, it is understood that each intervening value, to the tenth of the unit of the lower limit between the upper and lower limit of that range, and any other stated or intervening value in that stated range, is encompassed within the invention unless the context clearly dictates otherwise. The upper and lower limits of these smaller ranges may independently be included in the smaller ranges, and are also encompassed within the invention, subject to any specifically excluded limit in the stated range. Numerical ranges or amounts prefaced by the term “about” expressly include the exact range or exact numerical amount.

As used herein, a nucleic acid “sequence” means a nucleic acid base sequence of a polynucleotide. Unless otherwise indicated or apparent from context, bases or sequence elements are presented in the order 5′ to 3′ as they appear in a polynucleotide.

A “polynucleotide” or “nucleic acid” includes any form of RNA or DNA, including, for example, genomic DNA; complementary DNA (cDNA), which is a DNA representation of messenger RNA (mRNA), usually obtained by reverse transcription of mRNA; and DNA molecules produced synthetically or by amplification. Polynucleotides include nucleic acids comprising non-standard bases (e.g., inosine). A polynucleotide in accordance with the invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Polynucleotides may be single-stranded or double-stranded.

The term “oligonucleotide” is used herein to refer to a nucleic acid that is relatively short, generally shorter than 200 nucleotides, more particularly, shorter than 100 nucleotides or shorter than 70 nucleotides. Typically, oligonucleotides are single-stranded DNA molecules.

The term “segment” refers to a sequence or subsequence in a polynucleotide, such as a segment having a particular function, e.g., probe-binding segment, primer-binding segment, bar-code sequence, also referred to herein as a “zip code sequence”, and others listed herein. Individual segments may have any length consistent with their intended function, such as, without limitation, lengths in the range of 4-30 nucleotides.

As used herein, the term “complementary” refers to the capacity for precise pairing between two nucleotides. I.e., if a nucleotide at a given position of a nucleic acid is capable of hydrogen bonding with a nucleotide of another nucleic acid, then the two nucleic acids are considered to be complementary to one another at that position. A “complement” may be an exactly or partially complementary sequence. Two oligonucleotides are considered to have “complementary” sequences when there is sufficient complementarity that the sequences hybridize (forming a partially double stranded region) under assay conditions.

The terms “anneal”, “hybridize” or “bind,” in reference to two polynucleotide sequences, segments or strands, are used interchangeably and have the usual meaning in the art. Two complementary sequences (e.g., DNA and/or RNA) anneal or hybridize by forming hydrogen bonds with complementary bases to produce a double-stranded polynucleotide or a double-stranded region of a polynucleotide.

Two sequences or segments in a polynucleotide are “adjacent” or “contiguous” if there is no intervening sequence or non-nucleotide linker separating them.

A “primer” is an oligonucleotide or polynucleotide comprising a sequence that is complementary to, and capable of hybridizing to, a target sequence, or the complement thereof. In general, “primer” means an “extendible primer” that can prime template-dependent DNA synthesis.

The terms “multiplex” and “multiplexing” refer to assays in which two or more analytes are evaluated in the same reaction mixture. For example, a multiplex assay may comprise a plurality of proximity extension sets such that multiple analytes, e.g., multiple proteins, can be detected in the same reaction mixture.

As used herein, “amplification” of a nucleic acid sequence has its usual meaning, and refers to in vitro techniques for enzymatically increasing the number of copies of a target sequence. Amplification methods include both asymmetric methods (in which the predominant product is single-stranded) and conventional methods (in which the predominant product is double-stranded).

The terms “amplicon” and “amplification product” are used interchangeably and have their usual meaning in the art. The grammatically singular term, “amplicon,” can refer to many identical copies of an amplification product. Moreover, reference to an “amplicon” encompasses both a molecule produced in an amplification step and identical molecules produced in subsequent amplification steps (such as, but not limited to, amplification products produced in subsequent rounds of a PCR amplification). Moreover, the term “amplification may refer to cycles of denaturation, annealing and extension, and does not require geometric or exponential increase of a sequence.

A “amplification reaction mixture” is the solution in which an amplification reaction takes place and may comprise one or more of target polynucleotides, primers, polymerase, amplicons, amplification reagents, e.g., buffering agents, nuclease inhibitors, divalent cations, dNTPs, and/or other components known in the art for amplification.

An “extension reaction mixture” is a solution that contains products for template-directed DNA synthesis by a DNA polymerase and includes polymerase, dNTPs, divalent cations, buffering agents and other reagents known in the art for DNA synthesis.

As used herein, unless otherwise specified, the use of the term “antibody” encompasses a full-length Ig (including the constant regions) as well as a fragment of an antibody that retains antigen binding activity, e.g., a Fab, Fab′, F(ab′)₂, or scFv.

The term “qPCR” is used herein to refer to quantitative real-time polymerase chain reaction (PCR), which is also known as “real-time PCR” or “kinetic polymerase chain reaction.”

As used herein, a “sample” refers to a composition containing a polypeptide and/or polynucleotide analyte(s) of interest. In the present invention, a sample evaluated in a proximity extension assay of the invention is often a lysate from a single cell. The source of cells analyzed in accordance with the invention may be eukaryotic (e.g., from human, an animal, a plant, stem cells, blood cells, lymphocytes, yeast, fungi, or cells obtained from any plant or animal) or prokaryotic (e.g., bacterial, archaeal, or other prokaryotes). Cells analyzed using proximity extension assays and reagents as described herein include recombinant cells and cells infected with a pathogen. Examples of cells are explained in further detail below in section VIII.

A “reagent” refers broadly to any agent used in a reaction, other than the analyte (e.g., protein being analyzed). Illustrative reagents for a nucleic acid amplification or extension reaction include, but are not limited to, buffer, metal ions, polymeraseprimers, template nucleic acid, nucleotides, labels, dyes, nucleases, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

### I. Overview of Proximity Extension Assays

In one aspect, the invention provides proximity extension assay methods for detecting an analyte of interest in a sample, e.g., a sample from a single cell. Such methods of the invention provide an increase in assay sensitivity, e.g., by reducing the background and thus increasing the signal to background ratio.

The term “proximity extension assay” as used herein refers to an assay that employs a proximity extension probe set that has at least two members, where presence of the analyte target(s) of interest results in hybridization of oligonucleotide components of the probes. The hybridized probe product is extended and can then be detected. Proximity extension assays for detecting proteins are known in the art (see, for example, Lundberg et al. Nucl. Acids Res. 39: e102, 2011; WO2012/104261, and WO2013113699. each of which is incorporated by reference). A proximity extension probe comprises a region that binds to the analyte of interest linked to an oligonucleotide component that comprises a region that is complementary to a region of the oligonucleotide component of a second member of the probe set. The oligonucleotide component of the second member of the probe set is linked to a binding region (also referred to herein as “binding component”) that binds to either the same analyte at a site separate from the binding site for the first probe or a second analyte of interest. In typical embodiments, the analyte is a protein and the binding region is an antibody. Upon binding of the binding components of the probes to the analyte(s) of interest, the complementary oligonucleotides hybridize and are extended by a DNA polymerase in a reaction that comprises nucleotides, divalent cations, and other reagents for extending a primer. This results in a double-stranded DNA template that can be detected. In typical embodiments, the template is detected using quantitative PCR; however, a variety of other amplification systems may be used, as discussed below in section VII.

The invention provides proximity extension probes for use in detection of proteins and nucleic acids, e.g., in single cells. Proximity probes for use in the present invention are used in sets, typically in pairs. For detection of a protein of interest in a single cell sample, each probe typically comprises an antibody linked to an oligonucleotide. As noted above, the probe further comprises an oligonucleotide that contains a region that is complementary to a segment of the oligonucleotide of another member of the proximity probe set.

The methods of the invention can be conveniently used in a multiplex assay format. For example, if two or more target molecules, e.g., two or more target proteins, are to be detected, the products can be detected in a single reaction using multiple pairs of proximity probes, each of which forms an extension product that is unique. An assay of the invention can thus be readily multiplexed to evaluate the presence or amounts of multiple target molecules, e.g., proteins, in a sample.

Amplification primers are used to amplify the extended product resulting from hybridization of the oligonucleotide moieties of the proximity extension probes. The determination of the presence, absence, quantity, or relative amount of the amplified product is indicative of the presence, absence, quantity, or relative amount of the target analyte in the initial sample.

A proximity extension probe typically comprises DNA in an oligonucleotide component, but may also include polyribonucleotides (containing D-ribose), and any other type of nucleic acid that is an N- or C-glycoside of a purine or pyrimidine base, as well as other polymers containing normucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. The oligonucleotide component comprises an interacting region that binds to a complementary sequence on another proximity extension probe. The proximity extension probe further comprises a component that binds to a target of interest, e.g., a protein, in a sample. The binding component is often an antibody, either polyclonal or monoclonal, or fragment thereof, but also may be any other moiety that is capable of binding the target of interest, e.g., aptamers, a lectin, a soluble cell-surface receptor or derivative thereof, an affibody or any combinatorially derived protein or peptide from phage display or ribosome display or any type of combinatorial peptide or protein library. Combinations of any analyte-binding domain may be used.

Antibodies linked to each member of the protein proximity probe pair may have the same binding specificity or differ in their binding specificities. The present invention further contemplates use of variations of this assay, e.g., that are described in WO2012/104261. For example, the probes may each be linked to their respective antibody at the 5′ end, or one probe may be linked at the 5′ end and the other at the 3′ end.

The oligonucleotide segment is generally less than 70 nucleotides in length, and may be less than 50 or 45 nucleotides in length. As further detailed below, these ranges are illustrative guidelines but are not intended to limit the invention.

The interacting region of a proximity extension probe that interacts with a second member of the proximity extension probe set is located at or near the 3′ end of the probe such that the region is available to hybridize to the complementary sequence of the other member of the probe set when the proximity probes bind to an analyte, e.g., a protein. In typical embodiments, a hybridizing segment is designed such that upon hybridization with the interacting segment of the other member of the proximity pair, there are no 3′ non-base-paired nucleotides. However, other embodiments are also contemplated. For example, the 3′ end, i.e., that has the free 3′ hydroxyl group, of one of the proximity probes may not be included in segment that binds to the complementary segment of the other member of the proximity probe pair, thus leaving non-base-paired nucleotides at the 3′ end. Use of a polymerase having a 3′ exonuclease activity will permit the extension of the probe that has the 3′ non-based-paired nucleotides. In some embodiments, only one of the probes may be extended. For example, one of the probes may have a modified base at the 3′ end that prevents extension of the probe. In some embodiments, the 3′ nucleotide may be phosphorylated. In other embodiments, the 3′ end may have a modified nucleotide such as a thiophosphate-modified nucleotide, a 2′-OMe-CE phsophoramidite-modified nucleotide, or another extension-blocking nucleotide known in the art.

Typically, the interacting segment that interacts with the complementary region present on another member of the proximity probe set is often less than 20 or 15 nucleotides in length. For example, the interacting segment may be from 5 to 12 nucleotides in length, e.g., 6, 7, 8, 9, 10, 11 or 12 nucleotides in length.

Upon binding of the proximity probes to the target analyte and extension of the hybridized oligonucleotide components of the proximity probes, the extended product serves as a template for an amplification reaction

The extension reaction is performed at a temperature appropriate for the selected polymerase and under conditions in which the binding moieties, e.g., antibodies, remain bound to the target proteins such that the 3′ complementary ends of the probe pairs can hybridize. Similarly, in an assay in which at least one of the members of the probe set detects a nucleic acid moiety, the extension reaction is performed at a temperature appropriate for the selected polymerase and under conditions in which the oligonucleotide components of the proximity probes remain hybridized to one another.

As further explained below, in the present invention, e.g., using a proximity extension assay to evaluate analytes in a single cell, the extension reaction may be conducted after a separate step of incubation of the proximity extension probe set with the sample or at the same time as the step of incubating the probes with the sample. The extension reaction comprises reagents that are necessary for template-directed DNA synthesis. Such reagents include nucleotides as well as a polymerase. Any DNA polymerase can be used. In some embodiments the DNA polymerase lacks 3′ to 5′ exonuclease activity. In some embodiments, the the DNA polymerase has 3′ exonuclease activity. Examples of polymerases include T4 DNA polymerase, T7 DNA polymerase, Phi29 (ϕ29) DNA polymerase, DNA polymerase I, Klenow fragment of DNA polymerase I, Pyrococcus furiosus (Pfu) DNA polymerase, and Pyrococcus woesei (Pwo) DNA polymerase. In some embodiments, an RNA-dependent DNA polymerase can be employed.

In some embodiments, different polymerases are used for the PEA extension and PCR. In some embodiments, the PCR polymerase is Klenow fragment of DNA polymerase I, Phusion High Fidelity DNA polymerase (New England Biolabs), or Phi29 (1)29) DNA polymerase.

Further aspects of the invention are described in the following sections, II-VI.

### II. Increasing Sensitivity of a Proximity Extension Assays to Detect Analytes of Interest.

In one aspect, the invention provides a method of increasing the sensitivity of a proximity extension assay to detect an analyte of interest, e.g., a protein of interest. In some embodiments, the assay increases the sensitivity of a proximity extension assay performed using a single cell. As understood in the art, the method may also be employed where the sample to be analyzed is from more than one cell. Thus, for example, a single cell can be evaluated for the presence/level of an analyte of interest, such as a protein of interest, or 2, 3, 5, 10 or more cells, or samples comprising hundreds or thousands, or more, cells can be analyzed.

A sample comprising cells to be evaluated can be divided and spatially separated into single cells, or a desired number of cells, into a multiwell plate, tube, microarray, microfluidic device, or slide and the like to obtain a single cell (or the desired number of cells). The single cell is isolated in a buffer and can be lysed under desired conditions. The total reaction volume of a proximity extension assay of the invention can vary, e.g., depending on the vessel in which the assay is performed. Thus, the reaction can be performed in a droplet, a microfluidic chamber or channel, a tube, or a well.

In one aspect of the invention, the sensitivity of a proximity extension assay is increased by decreasing the background so that the signal to background ratio is increased. Decreased background in a proximity extension assay is conveniently measured by determining Cq levels during quantitative amplification of the extended product that results for hybridization of oligonucleotide components of a proximity probe set.

In the present invention, background can be evaluated by assessing the “Cq” under various conditions. As used herein, the term “Cq” refers to the quantification cycle or the cycle number where a signal, such as fluorescence, increases above the threshold in a quantitative PCR assay. In particular, it is the cycle number corresponding to the intersection between the amplification curve and the threshold line when signal is plotted against the cycle number on a logarithmic scale. Thus, the Cq value is the relative measure of the concentration of the target in a qPCR assay. In the context of the current invention, C_{q} and Cₜ are considered to be equivalent.

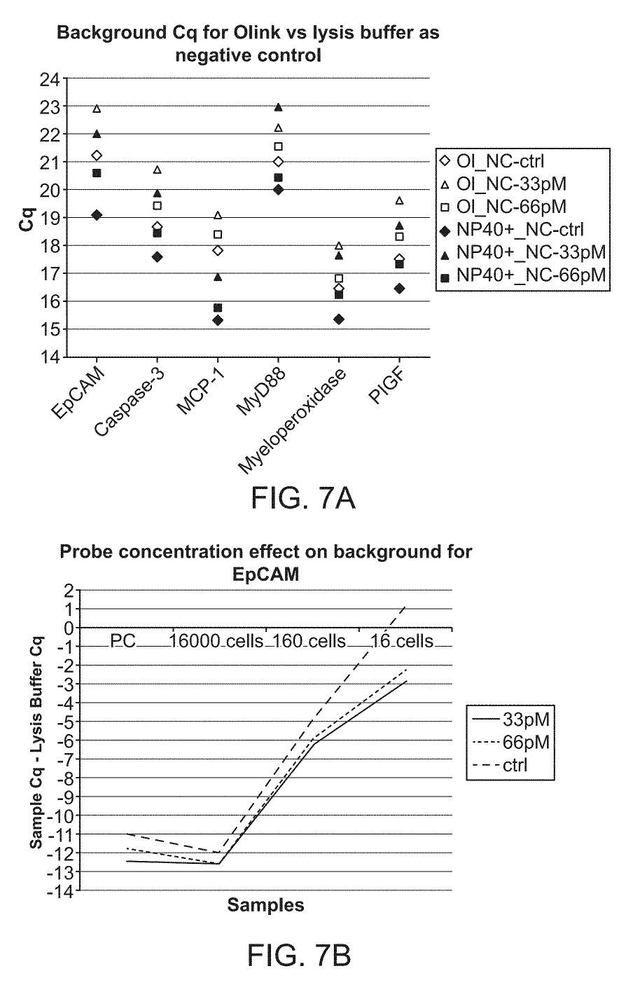

During the exponential amplification phase of a qPCR assay, the amount of the target template doubles with every cycle. Therefore, a Cq unit difference of 3 corresponds to a 2³ or 8 times change in the amount of the target. For instance, in FIG. 7A-7B, the difference in background Cq values for the EpCAM target between the Proseek negative control (e.g., C_{q} of about 21) and the 1% NP40 cell lysis buffer used according to the manufacture's recommendation (e.g., C_{q} of about 19) indicates that the 1% NP40 cell lysis buffer generates a background signal that is about 4 times (e.g., 2² times) higher than the negative control.

Thus, in an illustrative method of the invention, a single cell is isolated in an individual chamber on a microfluidics device. The cell is lysed in a solution that contains a surfactant, such as a detergent. Probes and extension reagents, which include a polymerase, nucleotides and other reagents necessary for DNA synthesis, are added. In some embodiments, the probes and/or extension reagents are added concurrently with the lysis solution. In some embodiments, probes and extension reagents are added after the cell has been lysed. Proximity extension assay characteristics that decrease background in accordance with the invention are described below.

### Surfactant Concentration

The lysis buffer contains a surfactant, typically a detergent, at a concentration that is below the critical micelle concentration (CMC), which is surfactant dependent. The CMC is the threshold concentration at which a surfactant aggregates in solution to form clusters (micelles). Because the formation of micelles from constituent monomers involves an equilibrium, the existence of a narrow concentration range for micelles, below which the solution contains negligible amounts of micelles and above which practically all additional surfactant is found in the form of additional micelles, has been established. A compilation of CMCs for hundreds of compounds in aqueous solution has been prepared by Mukerjee, P. and Mysels, K. J. (1971) Critical Micelle Concentrations of Aqueous Surfactant Systems, NSRDS-NBS 36. Superintendent of Documents, U.S. Government Printing Office, Washington, D.C. See also, http://www.anatrace.com/docs/detergent_data.pdf. CMC can be measured using known methods. For example, one technique used to determine CMC is direct measurement of equilibrium surface tension as function of surfactant concentration using a surface tensiometer. Other methods include measuring intensity of scattered light, solubilization of fluorescent dyes, etc., as a function of the surfactant concentration. These and other such techniques are well known in the art and are routinely employed.

In some embodiments, the lysis buffer contains a surfactant, typically a detergent, present at a concentration of 1.5% or less. In some embodiments, the surfactant is present in a range of from 0.01-1.0%. In some embodiments, the surfactant is present at a concentration of 1.5% or below, e.g., in a range of 0.1% to 1.5% or 0.1% to 1.0%. In some embodiments, the surfactant is present in a range of 0.05 to 0.5% or in a range of 0.1% to 0.25%. In some embodiments, a non-ionic detergent is employed, for example for analyses performed to identify protein-protein or protein-nucleic acid interactions. Use of surfactant, e.g., detergent, concentrations in the range of 0.01-0.5% can increase the sensitivity of a single cell protein analysis by reducing the background compared to using higher concentrations of detergent, such as greater than 1.5% detergent. In some embodiments, background in detecting a protein of interest in a single cell proximity extension assay is reduced by 2 to 3-fold when the sample is incubated in a buffer containing 0.1% detergent compared to a buffer containing 1.0% detergent.

Typical non-ionic detergents include the Triton series of detergents, e.g., Triton X-100 or TritonX-114; the Tween series, e.g., Tween 20 or Tween 40; NP-4; the Brij series of detergents, e.g., Brij-35 or Brij-58; or a glycoside, such as octylglucoside, octyl-thioglucoside, or a maltoside. Additional non-ionic detergents include alkylphosphine oxide (APO) non-ionic detergents such as Apo-12. Zwitterionic detergents, which possess a net zero charge arising from the presence of equal numbers of +1 and −1 charged chemical groups, can also be employed. Examples include CHAPS and CHAPSO.

In some embodiments, an ionic detergent, such as SDS, sodium cholate, or sodium deoxycholate, can be used.

In some embodiments, the lysis buffer may comprise additional components, such as a protease inhibitor.

In additional embodiments, the signal to noise ratio may be increased by including a denaturing step where the cell lysate is heated to reduce protein interaction.

### Probe Concentration

In some embodiments, increased sensitivity of a proximity extension assay in accordance with the invention may be achieved by using one or more proximity probes where the antibody has a binding affinity (as expressed by K_{d}) of 1 nM or lower, typically 100 pM or 10 pM or lower). In some embodiments, the antibody has a binding affinity in the range of about 1 pM to about 1 μM. some embodiments, the antibody has a binding affinity in the range of about 1 pM to about 500 nM or about 5 pM to about 500 nM. some embodiments, the antibody has a binding affinity in the range of about 10 pM to about 100 nM. some embodiments, the antibody has a binding affinity in the range of about 1 pM to about 500 pM. In some embodiments, the antibody has a binding affinity in the range of about 10 pM to about 100 pM. Thus, in some embodiments, e.g., where the antibodies used for the proximity probes are polyclonal antibodies, the probes are used at a concentration ranging from about 10 pM to about 200 pM, or in some embodiments about 10 pM to about 100 pM or about 20 to about 60 pM, in the binding step in which the proximity probes are incubated with the sample to allow binding of the probe to the analyte of interest, if present in the sample. In some embodiments, e.g., where the antibodies used for the proximity probes are monoclonal antibodies, proximity probes are employed at a concentration ranging from about 25 pM to about 1 nM or in some embodiments, a range of from about 50 pM to about 200 pM, in the binding step. In embodiments in which monoclonal and polyclonal antibodies are both present on proximity probes, the probes are typically employed at a concentration of range of between about 1 pM to about 250 pM or in some embodiments, at a range of about 10 pM to about 100 pM during the binding step.

In some embodiments, a proximity probe is provided at a concentration of from about 5 to about 250 pM, e.g., 10, 20, 30, 40, 50, 60, 70, 80, 90, 100, 150, or 250 pM. In some embodiments, the probe concentration in a single cell proximity extension assay is in the range of between about 75 pM and about 150 pM, or between about 50 pM and about 200 pM.

### Incubation

The sensitivity of a proximity extension assay can be enhanced by increasing the temperature of incubation of the probes with the sample and decreasing the incubation time. In some embodiments, the probes are incubated with the sample at a temperature ranging from about 15° C. to about 50° C. In some embodiment, incubation is at a temperature in the range from about 25° C. to about 50° C. In some embodiments, incubation is performed at a temperature ranging from about 25° C. to about 42° C. In some embodiments, incubation is performed at a temperature ranging from about 30° C. to about 40° C., e.g., at about 32° C., 33° C., 34° C., 35° C., 36° C., 37° C., or 38° C.

In typical embodiments where the proximity probe set is incubated with the sample at a temperature described above, the length of incubation of the probe set and sample is for a time period of ten hours or less, e.g., eight hours or less, or six hours or less, but for a time period greater than 2 minutes. In some embodiments, the incubation time period is about 3 hours, or about 2 hours, or less. In some embodiments, the incubation is performed for a length of time ranging from about 15 minutes to about six hours. In some embodiments, incubation is performed for a period ranging from about 30 minutes to about 3 hours, or from a time period ranging from about 30 minutes to about 2 hours, or for a period ranging from about 15 minutes to about 60 minutes.

As explained above, the proximity extension assay can be performed in separate steps in which the probe set is incubated with the sample and the polymerase and extension reagents are added following an initial incubation period as described above; or the incubation and extension steps can be combined into a singled step. In some embodiments, e.g., performing a single-cell proximity extension assay using a microfluidics device, the PEA extension polymerase enzyme and PCR polymerase are introduced, either separately or together, after an initial incubation period in which the probes are incubated with the sample. In some embodiments, the PEA probes can be combined with cell lysis. For example, a PEA analysis in a single cell microfluidics device may employ a lysis buffer containing a non-ionic detergent, e.g., 0.5% NP-40. The probe incubation step and the cell lysis step may be combined in the initial steps.

In some embodiments, increasing the incubation temperature above 4° C. as described here and decreasing the length of incubation of the probes with the sample to six hours or less can reduce the background by about 2-fold or greater. In some embodiments, combining increased incubation temperature and decreased incubation time in a sample lysate containing 0.5% or less, or 0.1% or less, non-ionic detergent and a probe concentration of 50 pM, or 30 pM or less, can decrease background by 2-fold or greater, e.g., 5-fold or greater, or 7 to 10-fold or greater.

In some embodiments, incubating PEA probes with the sample is performed at a temperature of about 30° C., or higher, e.g., from about 30° C. to about 40° C., for a period of time ranging from 30 minutes to 3 hours, e.g., about 1 to 2 hours.

### Exonuclease Step

In some embodiments, a proximity extension assay may include a step following probe incubation with the sample in which the annealed probes are incubated with an exonuclease that lacks polymerase activity, e.g., Exonuclease T or Exonuclease 1. For example, in a single-cell proximity extension assay using a microfluidics device, an exonuclease may be included in an incubation step with the annealed probes, e.g., to reduce the background. Alternatively, this may be accomplished using a polymerase that has exonuclease activity.

### Reaction Volume

The total volume of the reaction can vary depending on the reaction vessel. For example, in some embodiments, e.g., where the reaction vessel is a tube or a well, the incubation volume for the binding reaction in which the probes bind to the analyte of interest, if present, can be performed in the range of about 0.2 uL to about 150 uL, or in the range of about 0.2 uL to about 135 uL. In some embodiments, the incubation reaction for the binding reaction is in the range of about 1 uL to about 100 uL, or in the range of about 1 uL to about about 50 uL. In some embodiments, the incubation volume is in the range of about 1 uL to about 20 uL or about 1 uL to about 15 uL. In some embodiments, the incubation volume is less than any one of the following amounts: about 200 uL, about 150 uL, about 135 uL, about 120 uL, about 100 uL, about 75 uL, about 50 uL, about 25 uL, about 20 uL, about 15 uL, or about 10 uL, but greater than about 5 uL. The extension volume may also vary. The “extension volume” as used herein typically refers to the total volume of the reaction when the extension mixture is added with the binding reaction. Thus, in reactions in which the binding reaction and extension reactions are performed concurrently or performed consecutively where an extension mixture is added to the binding reaction, the extension reaction volume is the total reaction volume. For example, in some embodiments, the extension volume is in the range of from about 5 uL to about 500 uL. In some embodiments, the extension volume is the range of from about 10 uL to about 200 uL. In some embodiment, the extension volume is in the range of from about 20 uL to about 150 uL, or in the range of from about 10 uL to about 100 uL. In some embodiments, the extension volume is less than any one of the following amounts: about 500 uL, about 200 uL, about 170 uL, about 150 uL, about 100 uL, about 75 uL, about 50 uL, about 25 uL, or about 20 uL, but greater than about 5 uL.

in some embodiments, e.g., where the reaction vessel is chamber or a channel of a microfluidic device, the incubation volume for the binding reaction in which the probes bind to the analyte of interest, if present, can be performed in the range of about 0.2 nL to about 200 nL. In some embodiments, the incubation reaction for the binding reaction is in the range of about 1 nL to about 100 nL, or in the range of about 0.5 nL to about 50 nL. In some embodiments, the incubation volume is in the range of about 1 nL to about 20 nL or about 1 to about 15 nL. In some embodiments, the incubation volume is less than any one of the following amounts: about 200 nL, about 100 nL, about 50 nL, about 25 nL, about 10 nL, about 5 nL, or about 1 nL. The extension volume may also vary. For example, in some embodiments, the extension volume is in the range of from about 10 nL to about 10 uL. In some embodiments, the extension volume is the range of from about 10 nL to about to about 150 nL, or a range of from about 10 nL to about 150 nL. In some embodiment, the extension volume is in the range of from about 20 nL to about 150 nL. In some embodiments, the extension volume is less than any one of the following amounts: about 10 uL, about 5 uL, about 1 uL, about 500 nL, about 200 nL or about 150 nL, or less.

In some embodiments, for example when using a microfluidic device, the incubation volume of the binding reaction is 13.5 nL, 22.5 nL, 31.5 nL, or 166.5 nL. In some embodiments, the incubation volume of the extension reaction is 22.5 nL, 31.5 nL, 166.5 nL, or 301.5 nL.

In some embodiments, an initial PEA incubation and extension can be performed on one microfluidics device, the reactions harvested and the PCR performed on a second microfluidics device.

In some embodiments, a proximity assay in accordance with the invention may be performed in a droplet. In embodiments where droplets are preferred for the proximity extension assay, droplets may be formed by any method known in the art. The volume of droplet can be on the order of picoliters to nanoliters to microliters. Multiple droplets can be fused to bring reaction reagents into contact. In some embodiments, a sample droplet may contain a sample from a single cell. In some embodiments, the sample droplet may be combined with a lysis droplet containing a lysing buffer, e.g., a lysing buffer comprising a detergent present at a concentration below the critical micelle concentration, wherein a cell lysate is obtained by combining the sample and lysis droplets to form a cell lysate droplet. In some embodiments, the cell lysate droplet may be combined with a proximity extension probe droplet, e.g., a droplet containing two or more proximity extension probes, wherein the combined droplet may be incubated under any combination of incubation time and temperature detailed in section II to produce an incubation droplet wherein the proximity extension probes bind to the target analyte(s). In some embodiments, the incubation droplet may be combined with an extension reagent droplet, wherein the extension reagent droplet contains a polymerase to extend the hybridized oligonucleotide components of the proximity extension probes to produce extension products, to form an extension droplet. In some embodiments, the incubation droplet may be diluted according to the ratios detailed in section II before combining it with the extension reagent droplet. In some embodiments, the extension products are detected directly from the extension droplet.

In some embodiments, the proximity extension probe and extension reagent droplets may be combined to form a droplet, wherein that droplet is combined with the cell lysate droplet, at which point all steps of the proximity extension assay occur.

In some embodiments, the cell lysate, proximity extension probe and extension reagent droplets may all be combined concurrently to form an extension droplet, wherein all steps of the proximity extension assay occur.

Conversely, single droplets can be segregated from a larger body of liquid for subsequent treatment or interrogation. Additionally, a droplet can be combined with a larger body of liquid for subsequent treatment or interrogation. In some embodiments, the sample, lysing buffer, proximity extension probes and extension reagents may be contained in various separate liquid phases, e.g., fluid flows or droplets, of which at least one is contained in a droplet. A fluid flow can be combined with a droplet to produce a mixed fluid flow, a mixed droplet or both. In some embodiments, the various combinations of sample, lysis, cell lysate, proximity extension probe, incubation, extension reagent and/or extension droplets described above may be used wherein one or more of the droplets described in a particular embodiment is not contained in a droplet but rather another form of liquid, e.g., a fluid flow.

In general, smaller droplet volumes can be used with more sensitive detection methods. In some embodiments, for example, where a proximity extension assay of the invention is performed in a microfluidics device, the droplet has a diameter that is smaller than the diameter of the microchannel, e.g., preferably less than 60 microns. Thus, for example, in an embodiment with a channel of about 60 microns diameter, a typical free-flowing droplet is about 50 microns wide and 240 microns long. Droplet dimensions and flow characteristics can be influenced as desired, in part by changing the channel dimensions, e.g. the channel width. In some embodiments, the droplets of aqueous solution have a volume of approximately 0.1 to 100 picoliters (pl). Use of droplets for reactions is known in the art. Descriptions of droplet analysis using a microfluidics device are found, e.g., in U.S. patent application publication no. 20120276544 and Mazutis et al., Nature Protocols 8:870-891, 2013, which are incorporated by reference. Description of mixed droplet formation is found, e.g., in U.S. patent application publication no. 20120219947, which is incorporated by reference.

In an illustrative embodiment, a single cell is isolated and incubated in a surfactant-containing buffer that lyses the cell where the buffer contains the proximity probes. I some embodiments, reagents for extension of hybridized product (including polymerase and nucleotide reagents) may be included in the probe incubation buffer. The binding and extension steps are thus performed as a single step.

In alternative embodiments, the incubation mixture containing the proximity probes is added to the test samples in a binding reaction and incubated for a period of time as described above. The incubation mixture may added during cell lysis step or after the cells have been incubated with the lysis buffer. The extension mixing containing the extension polymerase and other extension reagents is then added following probe incubation. A polymerase for the PCR reaction may be included with an extension polymerase, or may be added to the incubation reaction separately. In some embodiments, the binding reaction mixture is diluted, e.g., at dilutions of from 1:2 to 1:20, or in some embodiments, 1:4 to 1:10, for prior to the addition of the polymerase and other extension reagents. In such embodiments the background signal can be reduced, for example, by anywhere from about 0.5 to about 10, or from about 0.5 to about 8 Ct, or from about 2 to about 6 Ct.

In one illustrative protocol, incubation mix containing proximity-DNA oligonucleotide probes at a concentration of 125 pM or less is added to a lysate from a single cell where the lysate was prepared using a buffer comprising 1.5% non-ionic detergent or less, e.g., 1.0% or less, or 0.5% or less, or 0.1% or less non-ionic detergent. After a 30 minute to one hour incubation at 37° C., an extension mix containing a DNA extension polymerase and extension reagents is added. After the extension period, extended products are analyzed using any suitable detection method, e.g., qPCR.

In some embodiments, one or more of the proximity probes is included in the lysis buffer. In some embodiments, one probe, e.g., a probe that has an antibody that has a higher affinity compared to another antibody in the proximity probe set, is added to the lysis buffer and the second probe is added following additional of the lysis buffer to the sample.

### III. Cells for Universal Proximity Extension Assay Positive Control

In a further aspect, the invention provides a universal positive control that can be used in proximity extension assays, e.g., proximity extension assays performed on a single cell. In some embodiments, proximity assays are performed using a surfactant concentration, temperature, length of incubation, probe concentration, and/or reaction volume detailed in Section II.

When interrogating single cell lysates, many proteins cannot be detected because common cell lines only expressed a portion of the human proteome. In addition, most proteins destined for secretion into the serum/plasma possess signal peptides that direct their export from cells directly into to the surrounding media and thus intracellular concentrations of such secreted proteins can be exceedingly low. In one aspect, the invention addressed the need for improved controls for proximity extension assays, e.g., proximity extension assays performed on a single cell.

In the present invention, thymic epithelial cells, e.g., human thymic epithelial cells, are used as a positive control. The thymus functions in the maturation process for the immune system T-cell population. An important requirement for proper immune system development is the elimination of T-cells that recognize self-antigens. Thymic epithelial cells play an important role in this function and possess promiscuous expression of mRNAs and their respective proteins. A large portion of the human proteome is expressed and displayed on the surface of TECs. (see, e.g., Magalhäes, et al., Clin Dev Immunol. 13:81-99, 2006; and Peterson et al., Nat Rev Immunol. 8:948-57, 2008). In some embodiments, thymic epithelial cells are employed as positive controls for proximity extension assay panels that detect serum or plasma proteins, or other secreted proteins.

In the present aspect, the invention thus provides thymic epithelial cells for use as a universal positive control for proximity extension assays. Thymic epithelial cells are known in the art and are commercially available. An example of a human TEC cell line is ATCC #CRL-7163 (human thymic epithelial cell line, HS202.TH, originally developed by the NBL repository—Naval Biosciences Laboratory). Other human TEC lines include those described in, e.g., Fernandez et al., Blood, 83(11): 3245-3254 (1994) can also be used in the methods provided herein. Protocols for culturing human TECs are described in detail in, e.g., Galy, A H, (1996). Methods in Molecular Medicine, 2:111-119, doi: 10.1385/0-89603-335-X:111 and Fernandez et al., Blood, 83(11): 3245-3254 (1994), which are incorporated by reference.

Thymic epithelial cells may be human or may be obtained from another animal, such as a mammal, e.g., rodent, such as rat or mouse thymic epithelial cells, or an avian. In addition to commercial sources, thymic epithelial cells can be obtained using well known methods. Protocols for culturing human TECs are described in detail in, e.g., Galy, A H, (1996). Methods in Molecular Medicine, 2:111-119, doi: 10.1385/0-89603-335-X:111 and Fernandez et al., Blood, 83(11): 3245-3254 (1994), which are incorporated by reference. For example, a thymic epithelial cell line may be cultured in standard media, such as DMEM supplemented with 10% fetal bovine serum. Cells can additionally be cultured under conditions to simulate the thymus microenvironment (see, e.g., Lee et al, J. Mater. Chem. 16:3558-3564, 2006).

In some embodiments, thymic epithelial cells are used as a positive control for proximity extension analysis performed on single cells. Thus, for example, a parallel sample of thymic epithelial cells are loaded onto a chamber, single cells from the sample are localized to individual attachment sites and the epithelial cells are monitored concurrently with the cells of interest. In some embodiments, thymic epithelial cells may be added to the target cell mixture and then loaded onto a chip for analysis.

In some embodiments, a lysate may be prepared from a large number so cells, e.g., 10³, 10⁴, 10⁵ cells, or more, and the lysate used in solution as a positive control for other assays, including assays conducted in a tube reaction or an immunoassay format. Such a lysate may also be used for single cell analysis.

In some embodiments, thymic epithelial cells are used for positive controls for analyzing RNA as well as protein.

### IV. Proximity Extension Assays to Evaluate Protein-Protein Interactions or Protein-Nucleic Interactions

In an additional aspect, the invention provides a method of detecting/quantifying protein-protein or protein-nucleic acid interactions using proximity extension assays. For example, such an analysis can be performed using a single cell. In this analysis, cells are subjected to a “gentle lysis” procedure that employs conditions that employ hypotonic buffer with very little or no detergent to preserve binding interactions. The proximity extension assays describe in this section can employ a surfactant concentration, incubation temperature, length on incubation, probe concentration, and/or reaction volume detailed in Section II.

In typical embodiments employing a gentle lysis procedure, non-shearing forces are used to mix the lysis reagent and isolated cell. The lysis buffer is typically a hypotonic buffer that contains a protein stabilization compound, such as a non-detergent sulfobetaine compound (e.g., NDSB-201, 195 or 256 at a concentration of 0.1%). A small amount, e.g., 0.01% to 0.05%, of a non-ionic detergent may also be included to facilitate lysis. In some embodiments, a lysis procedure is employed in which the nuclear membrane is preserved. In such a procedure, the cytoplasmic volume, as measured visually on a hemocytometer sizing grid, will typically increase by 10-40%, or 20-30%, for 50-100% or 80%-100% of the cells. Further, cell structures can be visually observed on an optical microscope slide without visible cell debris. In some embodiments, the cell is permeabilized where the cell membrane is porous, but still retains a structure.

In some embodiments, proximity extension probes may be directly introduced into cells, e.g., using patch clamp techniques or by direct injections. The cells may then be lysed to perform additional steps, such as the extension step and detection steps.

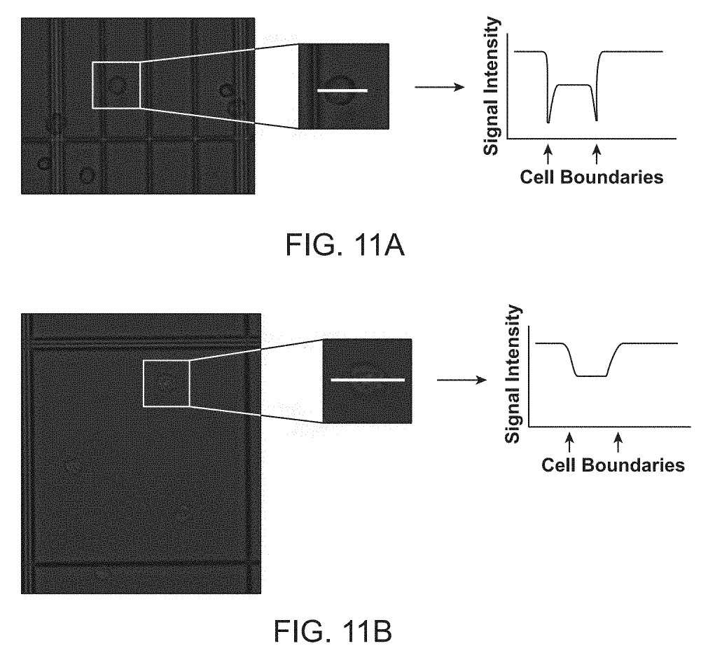

In an example of an assay to characterize gentle lysis of a cell(s), cell(s) are imaged on an optical microscope with image analysis capability before lysis. The greyscale microscope image is analyzed by plotting the signal intensity of a slice through the cell. The signal intensity plot will show sharp signal decreases at the cell boundaries, which represent reduced light penetrating the cytoplasmic membrane of the cell (FIG. 11A). The cell(s) are then mixed with a lysis reagent as described above. After a period of incubation, e.g., 5 minutes to 6 hours, the cell(s) are re-imaged on the microscope and the greyscale is again imaged by plotting the signal intensity of a slice through the cell. The signal intensity plot will show less or no sharp signal decreases at the cell boundaries (FIG. 11B) when the cytomembrane has been ruptured or permeabilized. Often, however, microscopic analysis will show cell structures, e.g., nuclei, are maintained.

In some embodiments, a lysis procedure is used that ruptures the cytoplasmic and nuclear membranes, but again preserves protein-protein and protein-nucleic acid binding interactions. For example, a non-ionic detergent such as NP40, Triton X-100 or Tween-20 may be added at 0.05-0.01% to the lysis buffer in addition to NDSB (at 0.1%). In this case, microscopic examination reveals fractured nuclei.

Gentle lysis procedures can also be modified depending on the original of the cell(s), e.g., whether the cell(s) are from a plant or animal or whether the cells are from a particular tissue.

Cells subjected to the lysis procedure can be incubated with proximity probes, either during lysis or following lysis. Incubation can be performed as described above. In some embodiments, extension reagents, including a polymerase and nucleotides, are added with the proximity probes. In some embodiments, extension reagents are added after an incubation period of the probes with the sample.

A proximity extension analysis of a cell(s) subjected to gentle lysis can be performed using a probe concentration, incubation temperature, length of incubation, and/or in a reaction volume as detailed in Section II.

In some embodiments, additional analyses, such as quantitative RT-PCR and/or whole genome amplification, can be performed using a reaction mixture following extension.

In some embodiments, both the proximity probes and cDNA may be extended with reverse transcriptase. In some embodiments, a protease is used to remove bound proteins from RNA prior to the RT reaction.

As noted above, proximity extension assays can be used to detect protein-protein interactions or protein-nucleic acid interactions. Thus, for example, in some embodiments, a proximity probe set is used where one probe comprises a protein-binding moiety, e.g, an antibody to a first protein of interest that participates in a protein-protein interaction linked to an oligonucleotide moiety comprising an interacting region and a second probe comprises a protein binding moiety, e.g., an antibody, that binds a second protein of interest that participates in the protein-protein interaction linked to an oligonucleotide that comprises an interacting region that is complementary to that of the interacting region of the first probe. When the proteins of interest are in a binding complex, binding of the probes allows for the formation of duplexes that can then be extended.

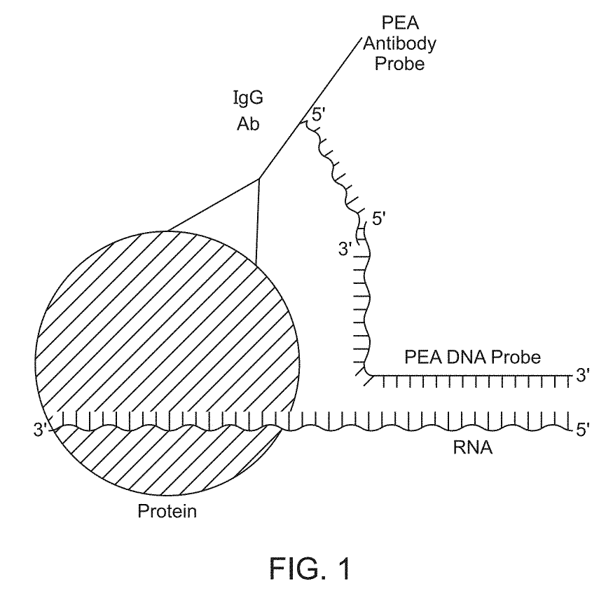

In some embodiments, the second probe is designed to bind to a nucleic acid, e.g., an RNA, to which the protein that is detected by the first probe binds. An illustration of such a probe combination is shown in FIG. 1.

### V. Proximity Extension Assay Configurations

Various modifications to proximity assay protocols as described herein may also be used. These include immobilization of one binding component of a proximity probe set to a solid phase and/or the use of 3 separate binding agents in a proximity probe set. These modifications can decrease background signal by 5 to 100-fold, often 10-50-fold. The assays described in this section can employ a surfactant concentration, incubation temperature, length on incubation, probe concentration, and/or reaction volume detailed in Section II.

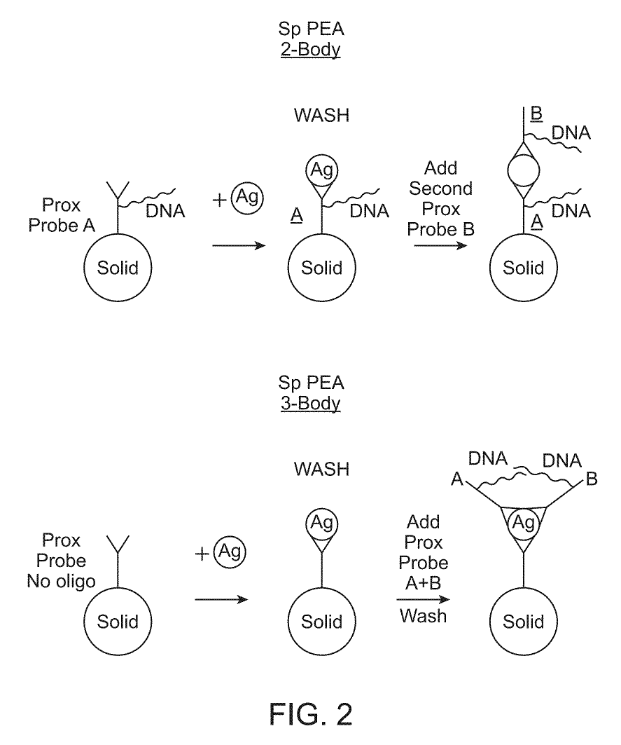

In one embodiment, one member of a proximity probe pair is immobilized on a solid phase, such as a bead or on the surface of the reaction vessel, e.g., on the surface of a microfluidic chamber or channel. This is illustrated in FIG. 2. For example, for detecting a target protein of interest, one member is immobilized to a solid surface and the sample is incubated with the immobilized binding moiety. In typical embodiments, the binding moiety is an antibody. This step can be followed by a wash step after which the second member of the proximity probe pair is incubated with the protein/proximity probe complex for performing a proximity extension assay.

In some embodiments, three binding moieties, typically three antibodies, can be employed, one of which is not contained in a proximity probe (see, FIG. 2). For example, an antibody may be attached to a solid surface and incubated with the antigen of interest. Following a wash step, a pair of proximity probes that also bind the antigen at different epitopes is added for performing a proximity extension assay.

As understood in the art, selection of parameters, e.g., probe concentration for the proximity extension assay, can vary depending on the configuration of the assay.

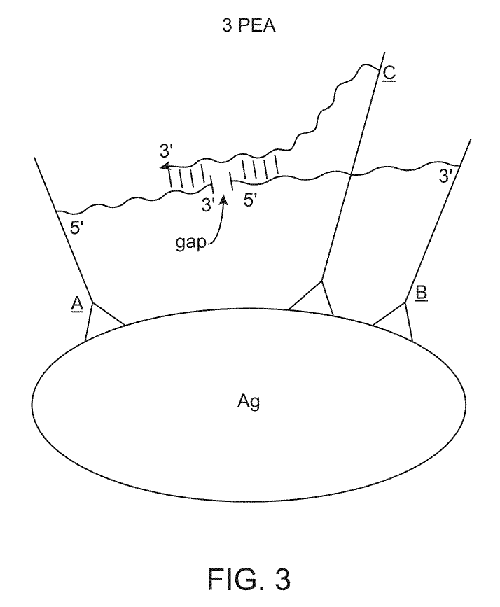

In some embodiments, a proximity probe set is used that comprises more than two members. For example, three probes can be used. For two of the probes, the oligonucleotide regions comprise the final amplicon sequence. The third probe has an oligonucleotide sequence (a “splint”) that facilitates hybridization of the other two oligonucleotide interacting regions. This is illustrated in FIG. 3. In this illustrative example, the 3′ end of one probe (probe C) hybridizes to both probes B and A. For example, probe B furnishes 5 nucleotides and Probe A the final 4 nucleotides. If all 9 nucleotides hybridize, the polymerase may extend through to the end of Probe A. If the oligonucleotide moiety of Probe B is not in proximity, Probe C cannot hybridize to Probe A. This design may also allow for a small gap, e.g., 1-5 nucleotides in Probe C between the regions where Probes A and B bind. In this configuration, Probe B is linked at its 3′ end to the antibody, whereas probes A and C are linked at their 5′ ends to the antibody. The sizes of regions of probes are not constrained by the sizes of the regions in FIG. 3 that illustrate an embodiment of the invention. The hybridizing regions are of sufficient length to maintain hybridization.

A binding moiety, e.g., an antibody, can be immobilized to a solid phase using well known techniques. In some embodiments, the antibody is immobilized to a bead. Suitable bead compositions may include plastics (e.g., polystyrene), dextrans, glass, ceramics, sol-gels, elastomers, silicon, metals, and/or biopolymers. Beads may have any suitable particle diameter or range of diameters, e.g, depending on the reaction vessel. Accordingly, beads may be a substantially uniform population with a narrow range of diameters, or beads may be a heterogeneous population with a broad range of diameters, or two or more distinct diameters. In some embodiments, the beads are of a size suitable for use in a microfluidic device, see, U.S. patent application Ser. No. 13/781,292 filed Feb. 28, 2013, which is incorporated by reference.

### VI. Alternative DNA Oligonucleotide Configuration—Two Sets of Complementary Sequences

In a further aspect, the invention provides a proximity extension assay that uses two sets of complementary sequences per proximity probe pair, instead of a single set of complementary sequences for each proximity probe pair. This configuration reduces background. The assays described in this section can employ a surfactant concentration, incubation temperature, length on incubation, probe concentration, and/or reaction volume detailed in Section II.

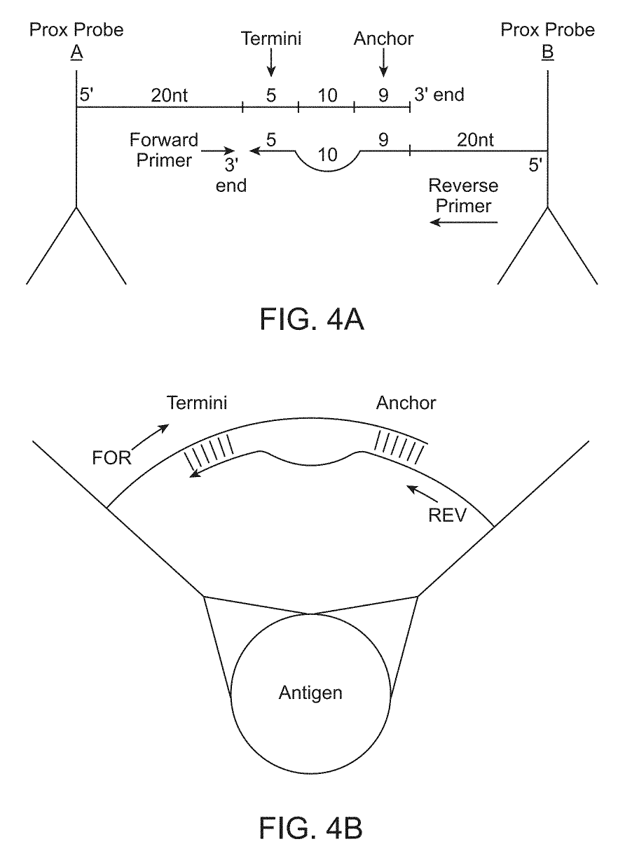

An example of oligonucleotide moieties present in proximity probe pairs that provide two hybridization sets of hybridization sequences is illustrated in FIG. 4A. In the embodiment illustrated in FIG. 4A, each 44-mer oligonucleotide contains an anchor motif of 6-9 nucleotides to connect the two proximity probes, a 10-nucleotide spacer and a 4-6 nucleotide motif at the termini. The motif at the terminal regions of the oligonucleotide only needs to meet the minimum DNA polymerase footprint requirements. Thus, the regions of an oligonucleotide component of a first member of a proximity pair can be described as follows, 5 to 3′: a forward primer binding site, an anchor sequence, a spacer, and a terminal sequence. The other member of the proximity probe pair comprises (5′ to 3′): a primer binding site for a reverse primer, a region that is complementary to the anchor sequence on the first oligonucleotide, a spacer, and a terminal region that is complementary to the terminal region of the first oligonucleotide.

In this configuration, the anchor complementary sequences are in close proximity to the antibody. The total length of the oligonucleotide component is typically in the range of 28 to 62 nucleotides. In some embodiments, the oligonucleotides are in the range of 36 to 51 nucleotides. In some embodiments, the oligonucleotides are from 42 to 48 nucleotides in length. The segments within the oligonucleotide may vary from the illustrative size shown in FIG. 4A. In some embodiments, the size of the segment containing the primer binding site (the region between the antibody and anchor segment) is in the range of 16-24 nucleotides. In some embodiments, the segment is 18-22 nucleotides in length. The anchor segment is typically 5-10 nucleotides in length. In some embodiments, the anchor region is 6 to 9 nucleotides in length. The spacer between the anchor segment and terminal segment can be anywhere from 5-20 nucleotides long. In typical embodiments, the spacer is from 8 to 14 nucleotides long, for example, 10 to 12 nucleotides long. As noted above, the terminal segment can be short, for example, 2 to 8 nucleotides long. In typical embodiments, the terminal binding segment is 4 to 6 nucleotides.

In some embodiments, a proximity probe pairs as described above is used in a proximity extension assay where the proximity probes, polymerase and other extension reagents are added to the reaction mixture at the same time, for example in an incubation for 5-30 minutes at 37° C. An example of the resulting structure is shown in FIG. 4B.

### VII. Amplification and Detection of Amplified Products

The extended products obtained from any of the extension reactions employing reactions conditions and/or probes as described in sections I to VI are subjected to an amplification reaction to obtain an amplified product that can be detected and quantified, as desired. Design parameters of various amplification reactions are well known. Examples of references providing guidance are provided below. In some embodiments the amplification reaction uses the same polymerase that is used in the extension assay, optionally without addition of more polymerase. In some embodiments the amplification reaction uses a polymerase that is different from the polymerase used for the extension assay. For example, in some embodiments, a polymerase having a 3′ exonuclease activity may be used in the extension reactions and a Taq polymerase may be used in the amplification reaction.

In some embodiments, an amplification reaction may employ a hot-start polymerase. For example, a recombinant Taq DNA polymerase complexed with an antibody that inhibits polymerase activity at ambient temperatures may be used. The polymerase is active after a PCR denaturation step.

Any method of detection and/or quantitation of nucleic acids can be used in the invention to detect and/or quantify amplification products. In particular embodiments, real-time quantification methods are used. For example, “quantitative real-time PCR” methods can be used to determine the quantity of an amplified product present in a sample by measuring the amount of amplification product formed during the amplification process itself. This method of monitoring the formation of amplification product involves the measurement of PCR product accumulation at multiple time points. The amount of amplified product reflects the amount of target nucleic acid or target protein present in the sample.

Fluorogenic nuclease assays are one specific example of a real-time quantitation method that can be used successfully in the methods described herein. This method of monitoring the formation of amplification product involves the continuous measurement of PCR product accumulation using a dual-labeled fluorogenic oligonucleotide probe—an approach frequently referred to in the literature as the “TaqMan® method.” See U.S. Pat. No. 5,723,591; Heid et al, 1996, Real-time quantitative PCR Genome Res. 6:986-94, each incorporated herein by reference in their entireties for their descriptions of fluorogenic nuclease assays. It will be appreciated that while “TaqMan® probes” are the most widely used for qPCR, the invention is not limited to use of these probes; any suitable probe can be used.

Other detection/quantitation methods that can be employed in the present invention include FRET and template extension reactions, molecular beacon detection, Scorpion detection, and Invader detection.

FRET and template extension reactions utilize a primer labeled with one member of a donor/acceptor pair and a nucleotide labeled with the other member of the donor/acceptor pair. Prior to incorporation of the labeled nucleotide into the primer during a template-dependent extension reaction, the donor and acceptor are spaced far enough apart that energy transfer cannot occur. However, if the labeled nucleotide is incorporated into the primer and the spacing is sufficiently close, then energy transfer occurs and can be detected. These methods are described in U.S. Pat. No. 5,945,283 and PCT Publication WO 97/22719.

With molecular beacons, a change in conformation of the probe as it hybridizes to a complementary region of the amplified product results in the formation of a detectable signal. The probe itself includes two sections: one section at the 5′ end and the other section at the 3′ end. These sections flank the section of the probe that anneals to the probe binding site and are complementary to one another. One end section is typically attached to a reporter dye and the other end section is usually attached to a quencher dye. In solution, the two end sections can hybridize with each other to form a hairpin loop. In this conformation, the reporter and quencher dye are in sufficiently close proximity that fluorescence from the reporter dye is effectively quenched by the quencher dye. Hybridized probe, in contrast, results in a linearized conformation in which the extent of quenching is decreased. Thus, by monitoring emission changes for the two dyes, it is possible to indirectly monitor the formation of amplification product. Probes of this type and methods of their use are described further, for example, by Piatek et al. (1998) Nat. Biotechnol. 16: 359-363; Tyagi, and Kramer (1996) Nat. Biotechnol, 14: 303-308; and Tyagi, et al. (1998) Nat. Biotechnol. 16:49-53. [0124] The Scorpion detection method is described, for example, by Thelwell et al. (2000) Nucleic Acids Res., 28: 3752-3761 and Solinas et al. (2001) Nucleic Acids Res., 29(20): e96. Scorpion primers are fluorogenic PCR primers with a probe element attached at the 5′-end via a PCR stopper. They are used in real-time amplicon-specific detection of PCR products in homogeneous solution. Two different formats are possible, the “stem-loop” format and the “duplex” format. In both cases the probing mechanism is intramolecular. The basic elements of Scorpions in all formats are: (i) a PCR primer; (ii) a PCR stopper to prevent PCR read-through of the probe element; (iii) a specific probe sequence; and (iv) a fluorescence detection system containing at least one fluorophore and quencher. After PCR extension of the Scorpion primer, the resultant amplicon contains a sequence that is complementary to the probe, which is rendered single-stranded during the denaturation stage of each PCR cycle. On cooling, the probe is free to bind to this complementary sequence, producing an increase in fluorescence, as the quencher is no longer in the vicinity of the fluorophore. The PCR stopper prevents undesirable read-through of the probe by Taq DNA polymerase.

As noted above, various amplification and reaction methods may be used to detect the extended product. Thus, amplification according to the present invention encompasses any means by which at least a part of the extended product is copied, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(I):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18- (2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2): 165-74, Schweitzer et al., Curr Opin Biotechnol. 2001 February; 12(I):21-7, U.S. Pat. Nos. 5,830,711, 6,027,889, 5,686,243, PCT Publication No. WO0056927A3, and PCT Publication No. WO9803673A1.

Amplification methods to detect extension products generated in a proximity extension assay in accordance with the invention include isothermal amplification methods. Isothermal amplification uses non-denaturing conditions for the amplification reaction. Some means of strand separation, e.g., an ezyme, is used in place of thermal denaturation. Examples of isothermal amplification include: hyperbranched strand displacement amplification (Groathouse, N., et al. (2006) “Isothermal Amplification and Molecular Typing of the Obligate Intracellular Pathogen Mycobacterium leprae Isolated from Tissues of Unknown Origins” J. Clin. Micro. 44 (4): 1502-1508); helicase-dependent amplification (Vincent, M., et al. (2004) “Helicase-dependent isothermal DNA amplification” EMBO Rep. 5 (8): 795-800); multiple displacement amplification (MDA; Luthra, R., and Medeiros, J. (2004) “Isothermal Multiple Displacement Amplification” J Mol Diagn. 6 (3): 236-242); loop-mediated isothermal amplification (Notomi, T., et al. (2000) Nucleic Acids Research 28 (1); PAN-AC (David, F. and Turlotte, E., (1998) “An Isothermal Amplification Method” C.R. Acad. Sci Paris, Life Science 321 (1): 909-14); strand displacement amplification (SDA; Nycz, C, et al. (1998) Analytical Biochemistry 259 (2): 226-234); rolling circle amplification (RCA; Lizardi, P., et al., (1998)“Mutation detection and single-molecule counting using isothermal rolling-circle amplification” Nature Genetics 19: 225-232); nucleic acid strand-based amplification (NASBA; Van Der Vliet, G., et al. (1993) “Nucleic acid sequence-based amplification (NASBA) for the identification of mycobacteria” Journal of General Microbiology 139 (10): 2423-2429; and recombinase polymerase amplification (U.S. Pat. Nos. 7,485,428; 7,399,590; 7,270,981; and 7,270,951, each of which is incorporated by reference in its entirety and specifically for its description of recombinase polymerase amplification).

In embodiments in which fluorophores are used as labels, many suitable fluorophores are known. Examples of fluorophores that can be used include, but are not limited to, rhodamine, cyanine 3 (Cy 3), cyanine 5 (Cy 5), fluorescein, Vic™, Liz™, Tamra™ 5-Fam™ 6-Fam™ and Texas Red (Molecular Probes). (Vic™, Liz™, Tamra™ 5-Fam™ 6-Fam™ are all available from Applied Biosystems, Foster City, Calif.).

In embodiments in which quenchers are also used for detection of amplified products, useful quenchers include, but are not limited to tetramethylrhodamine (TAMRA), DABCYL (DABSYL, DABMI or methyl red) anthroquinone, nitrothiazole, nitroimidazole, malachite green, Black Hole Quenchers®, e.g., BHQ1 (Biosearch Technologies), Iowa Black® or ZEN quenchers (from Integrated DNA Technologies, Inc.), TIDE Quencher 2 (TQ2) and TIDE Quencher 3 (TQ3) (from AAT Bioquest).

PCR and fluorescence detection are detected using systems well known in the art. For example detection can be performed using a system such as the BioMark™ System (Fluidigm Corporation, South San Francisco).

### VIII. Samples

Numerous analytes of interest can be detected using the proximity extension probe assays of the invention. In typical embodiments, the target analyte is a an antigen to which an antibody binds, e.g., a protein antigen In some embodiments, e.g., when protein-nucleic acid interactions are analyzed, a target analyte is a single-stranded nucleic acid, such as an RNA. The analytes to be evaluated, e.g., in analyzing a single cell, include, but are not limited to, proteins and nucleic acids associated with pathogens, such as viruses, bacteria, protozoa, or fungi; proteins for which over- or under-expression is indicative of disease, proteins that are expressed in a tissue- or developmental-specific manner; or analytes that are induced by particular stimuli.

Samples to be analyzed, including cells for single cell analysis, can be obtained from biological sources and prepared using conventional methods known in the art. In particular, samples to be analyzed in accordance with the methods described herein obtained from any source, including bacteria, protozoa, fungi, viruses, organelles, as well higher organisms such as plants or animals, particularly mammals, and more particularly humans. Other samples can be obtained from environmental sources (e.g., pond water, air sample), from man-made products (e.g., food), from forensic samples, and the like. Samples can be obtained from cells, bodily fluids (e.g., blood, a blood fraction, urine, etc.), or tissue samples by any of a variety of standard techniques. Illustrative samples include samples of plasma, serum, spinal fluid, lymph fluid, peritoneal fluid, pleural fluid, oral fluid, and external sections of the skin; samples from the respiratory, intestinal genital, and urinary tracts; samples of tears, saliva, blood cells, stem cells, or tumors. For example, samples can be obtained from an embryo or from maternal blood. Samples can also be obtained from live or dead organisms or from in vitro cultures. Illustrative samples can include single cells, paraffin-embedded tissue samples, and needle biopsies.

In some embodiments, the assays of the invention are conducted on single cells. In some embodiments, an assay is performed using a small number (e.g., fewer than 100, fewer than 50, fewer than 10, or fewer than 5) of cells. In one approach employing a single cell, the cell is isolated and lysed; and reagents, e.g., proximity extension probes, extension reagents, polymerases, amplification reagents are added directly to the lysate to perform the detection assay. In some embodiments, the isolation of single cells and proximity extension assay of the invention is carried out using a microfluidic device. Microfluidic systems for are known. An exemplary device is the C1™ Single-Cell Auto Prep System which is commercially available from Fluidigm Corp. 7000 Shoreline Court, Suite 100, South San Francisco, Calif.). The C1™ Single-Cell Auto Prep System isolates single cells, lyses them, and carries out a series of reactions from the lysate (e.g., cDNA synthesis, nucleic acid amplification, etc.). Other devices are described in U.S. patent application Ser. No. 13/781,292 filed Feb. 28, 2013, entitled “Methods, Systems, And Devices For Multiple Single-Cell Capturing And Processing Using Microfluidics”, which is incorporated by reference in its entirety for all purposes. Optionally the C1™ Single-Cell Auto Prep System may be used in conjunction with Fluidigm's BioMark™ HD System (Fluidigm Corp. 7000 Shoreline Court, Suite 100, South San Francisco, Calif.). U.S. patent application Ser. No. 13/781,292 filed Feb. 28, 2013 is incorporated herein in its entirety all purposes.

Single-cell studies within micro fluidic architectures may involve the isolation of individual cells into individual reaction partitions (chambers, droplets, cells). Limiting dilution is one method for achieving this isolation. Cells may be loaded at concentrations of less than one cell per partition on average, and distribute into those partitions in a pattern described by Poisson statistics. Another approach is to rely on mechanical traps to capture cells. These traps are designed to capture cells of a given size range.

Other devices for manipulation of single cells include the following: Sims et al., 2007, “Analysis of single mammalian cells on-chip” Lab Chip 7:423-440; Wheeler et al., 2003, “Microfluidic device for single-cell analysis” Anal Chem 75:3581-3586; Skelley et al., 2009 “Microfluidic control of cell pairing and fusion” Nat Methods 6:147-152; Marcus et al., 2006, “Microfluidic single-cell mRNA isolation and analysis” Anal Chem 78:3084-3089; Bontoux et al., 2008 “Integrating whole transcriptome assays on a lab-on-a-chip for single cell gene profiling” Lab Chip 8:443-450; Zhong et al., 2008 “A microfluidic processor for gene expression profiling of single human embryonic stem cells” Lab Chip 8:68-74; Wheeler 2003 “Microfluidic Device for Single-Cell Analysis Anal. Chem.” 75:3581-3586; and White et al., Aug. 23, 2011 “High-throughput microfluidic single-cell RT-qPCR PNAS” Vol. 108, 34:13999-14004; each of the aforelisted publications is incorporated herein by reference.

Additional methods for amplifying and detecting amplified products are described in U.S. Pat. Pub. Nos. 2012-0115143 (“Universal Probe Assay Methods”), US 2012-0288857 (“Multifunctional Probe-Primers”), US 2013-0045881 (“Probe Based Nucleic Acid Detection”); and copending commonly owned International Patent Application No. PCT/US2012/065376 (“NUCLEIC ACID DETECTION USING PROBES”) and International PCT Application No. PCT/US2007/063229 (“COOPERATIVE PROBES AND METHODS OF USING THEM”),each of which is expressly incorporated by reference for all purposes.

Cells for single cell analysis can be obtained from eukaryotic or prokaryotic organisms. Eukaryotics cells may be from animals, that is, vertebrates or invertebrates. Vertebrates may include mammals, that is, primates (such as humans, apes, monkeys, etc.) or nonprimates (such as cows, horses, sheep, pigs, dogs, cats, rabbits, mice, rats, and/or the like). Nonmammalian vertebrates may include birds, reptiles, fish, (such as trout, salmon, goldfish, zebrafish, etc.), and/or amphibians (such as frogs of the species Xenopus, Rana, etc.). Invertebrates may include arthropods (such as arachnids, insects (e.g., Drosophila), etc.), mollusks (such as clams, snails, etc.), annelids (such as earthworms, etc.), echinoderms (such as various starfish, among others), coelenterates (such as jellyfish, coral, etc.), porifera (sponges), platyhelminths (tapeworms), nemathelminths (flatworms), etc.

Eukaryotic cells may be from any suitable plant, such as monocotyledons, dicotyledons, gymnosperms, angiosperms, ferns, mosses, lichens, and/or algae, among others. Exemplary plants may include plant crops (such as rice, corn, wheat, rye, barley, potatoes, etc.), plants used in research (e.g., Arabadopsis, loblolly pine, etc.), plants of horticultural values (ornamental palms, roses, etc.), and/or the like.

Eukaryotic cells may be from any suitable fungi, including members of the phyla Chytridiomycota, Zygomycota, Ascomycota, Basidiomycota, Deuteromycetes, and/or yeasts. Exemplary fungi may include Saccharomyces cerevisiae, Schizosaccharomyces pombe, Pichia pastoralis, Neurospora crassa, mushrooms, puffballs, imperfect fungi, molds, and/or the like.

Eukaryotic cells may be from any suitable protists (protozoans), including amoebae, ciliates, flagellates, coccidia, microsporidia, and/or the like. Exemplary protists may include Giardia lamblia, Entamoeba. histolytica, Cryptosporidium, and/or N. fowleri, among othe

Eukaryotic cells for analysis may also be immortalized and/or transformed by any suitable treatment, including viral infection, nucleic acid transfection, chemical treatment, extended passage and selection, radiation exposure, and/or the like. Such established cells may include various lineages such as neuroblasts, neurons, fibroblasts, myoblasts, myotubes, chondroblasts, chondrocytes, osteoblasts, osteocytes, cardiocytes, smooth muscle cells, epithelial cells, keratinocytes, kidney cells, liver cells, lymphocytes, granulocytes, and/or macrophages, among others. Exemplary established cell lines may include Rat-1, NIH 3T3, HEK 293, COS 1, COS7, CV-1, C2C12, MDCK, PC12, SAOS, HeLa, Schneider cells, Junkat cells, SL2, and/or the like.

Prokaryotic cells that can be analyzed in accordance with the invention include self-replicating, membrane-bounded microorganisms that lack membrane-bound organelles, or nonreplicating descendants thereof. Prokaryotic cells may be from any phyla, including Aquificae, Bacteroids, Chlorobia, Chrysogenetes, Cyanobacteria, Fibrobacter, Firmicutes, Flavobacteria, Fusobacteria, Proteobacteria, Sphingobacteria, Spirochaetes, Thermomicrobia, and/or Xenobacteria, among others. Such bacteria may be gram-negative, gram-positive, harmful, beneficial, and/or pathogenic. Exemplary prokaryotic cells may include E. coli, S. typhimurium, B. subtilis, S. aureus, C. perfiingens, V. parahaemolyticus, and/or B. anthracis, among others.

### IX. Kits

Kits according to the invention include one or more reagents useful for practicing one or more assay methods of the invention. A kit generally includes a package with one or more containers holding the reagent(s) (e.g., a proximity extension probe set), as one or more separate compositions. In some embodiments, the probes may be provided as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay. In some embodiments, the kit may include a positive control, e.g., an extract from thymic epithelial cells.

Kits according to the invention generally include instructions for carrying out one or more of the methods of the invention. Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), RF tags, and the like. As used herein, the term “instructions” can include the address of an internet site that provides the instructions.

### EXAMPLES

These examples illustrate various aspects of the invention that provide for enhanced sensitivity of a proximity extension assay, e.g., for single cell analysis.

### Example 1. Proximity Extension Assay for Evaluating Analytes Present in a Single Cell

Proximity extension assays have previously been described using plasma and serum samples as input material (Lundberg et al., supra), which contains high amounts of proteins. This high level of protein generates signal that can be clearly distinguished from the high background signals detected when using the original method described by Lundberg (background Cq range for 92 protein target assays in the commercially available Olink Proseek kit is between 10-20). In one aspect, the present invention provides methods of increasing the sensitivity of a proximity extension assay that are suitable for evaluating analytes present in a single cell or in an extract from a small number of cells, e.g., less than 100 or 50 cells, or less than 20 cells.

Samples used: Cell lysates, instead of plasma or serum samples, were analyzed. A commercially available NP40 Cell Lysis Buffer suitable for the preparation of cell extracts to be analyzed by Antibody Bead Immunoassay (Luminex), ELISA, and Western blotting was used (Life Technologies, PN FNN0021). This buffer contains a non-ionic detergent (NP40) which at relatively high concentrations (e.g., 1%) may promote proximity probe aggregation in buffer solutions.

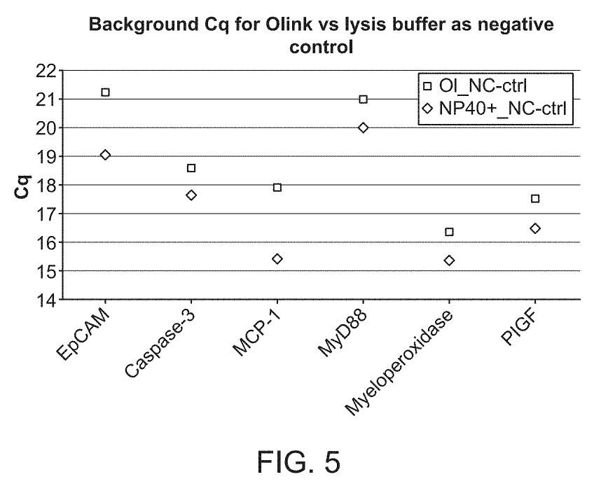

Increased background (1-3 Cq units) was observed when we tested the NP40 lysis buffer as per manufacturer's recommendation (1% concentration when compared to the Olink Proseek kit negative control (FIG. 5).

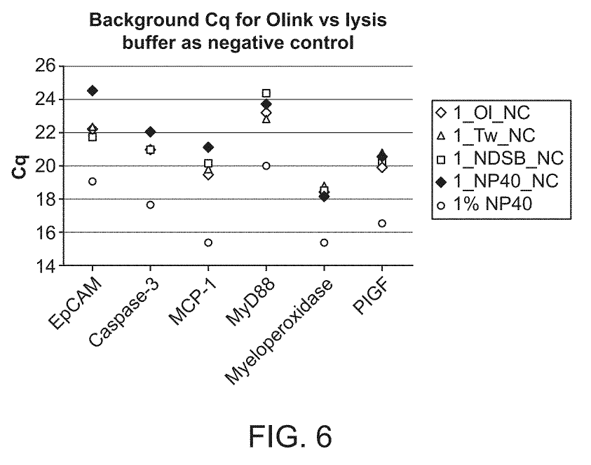

Lower concentrations of NP40 as well as non-ionic detergent alternatives as lysis buffers were tested (Tween-20 and Triton X-100). All 3 detergents were tested at 0.1% concentration and were effective in lysing cells (data not shown) while keeping the same background signal level as the Proseek kit negative control (FIG. 6).

### Probe Concentration for the Incubation Step:

While the method of Lundberg et al. calls for a final probe concentration of 100 pM in the incubation step, we found that the use of lower probe concentrations allowed signal distinction between 12 cells and background, which was not seen when using a 100 pM probe concentration (FIG. 7A-7B). This occurred despite the fact that the lower levels of protein in single cell lysate (˜300 pg) as compared to plasma or serum may lead one to expect that an increase in probe concentration would be needed to allow detection at this level.

### Length and Temperature of Incubation Period:

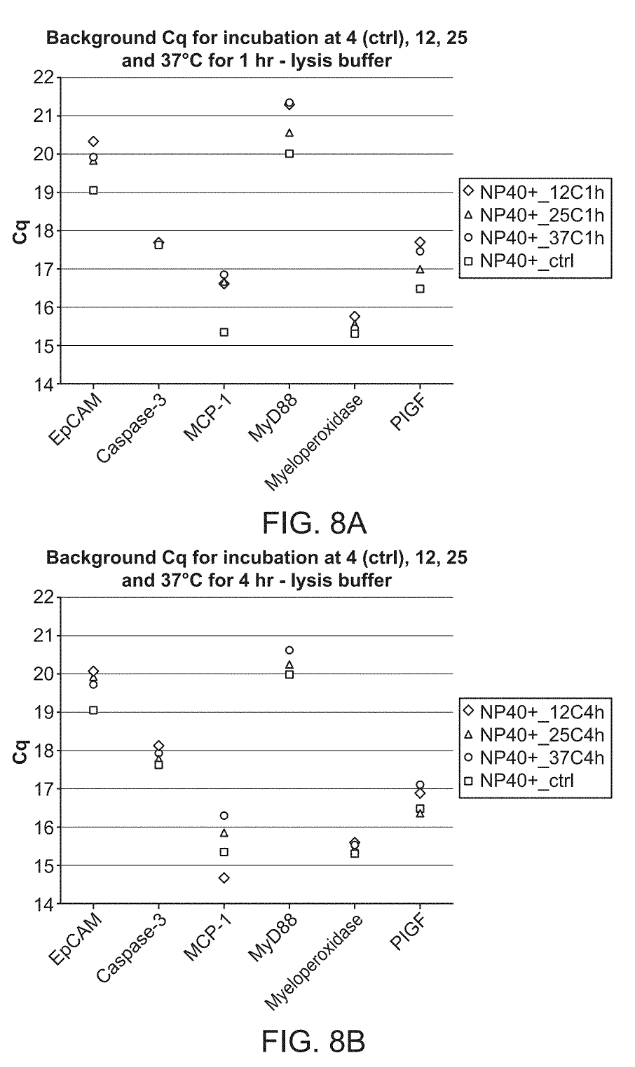

In additional experiments, we reduced the analyte binding times and increased incubation temperature. Reduced analyte binding incubation times minimize formation of proximity probe aggregrates. With probes constructed using high affinity antibodies and using unfractionated cell lysates, we reasoned that the length of the probe binding step could be shortened to 10-20 minutes. After this equilibrium time point is reached, the antibody binding on-off kinetic rates dominate the steady state levels of bound antibody. Therefore, we evaluated shorter incubation time periods, modifying the original protocol from 12-16 hours incubation to 4 and 1 hr. Additionally, since antibody-antigen interactions generally occur most favorably at 37° C., we also tested higher incubation temperatures (12, 25 and 37° C.) than the original protocol recommends (4° C.). The results showed that incubation at 4° C. for 12-16 hr caused the highest background for all assays tested (n=6) and 37° C. produced the most robust decrease of background for EpCAM in all cell input levels (FIG. 8A-8B).

### Extension Master Mix Preparation:

After incubation, the full solution of sample and incubation master mix (extension reaction template) is added to the extension master mix for the extension reaction. We evaluated the dilution of the extension template prior to polymerase addition to reduce non-proximal interactions and thus, background signals. This hypothesis can be characterized by the equation:

P=K[proximity probe 1]×[proximity probe 2], where

P=background polymerase extension product amount
K=a reaction constant number

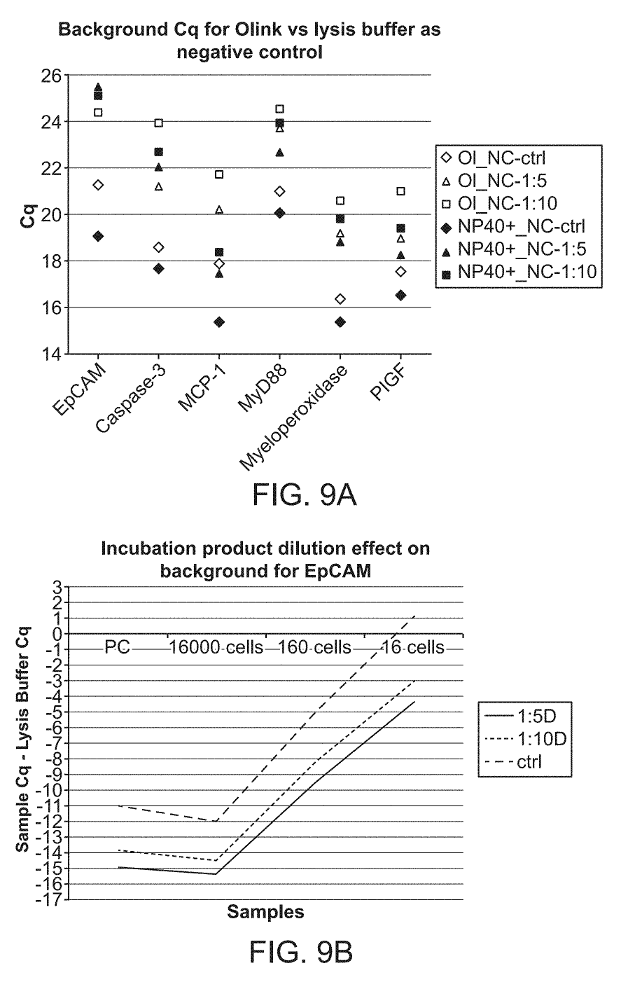

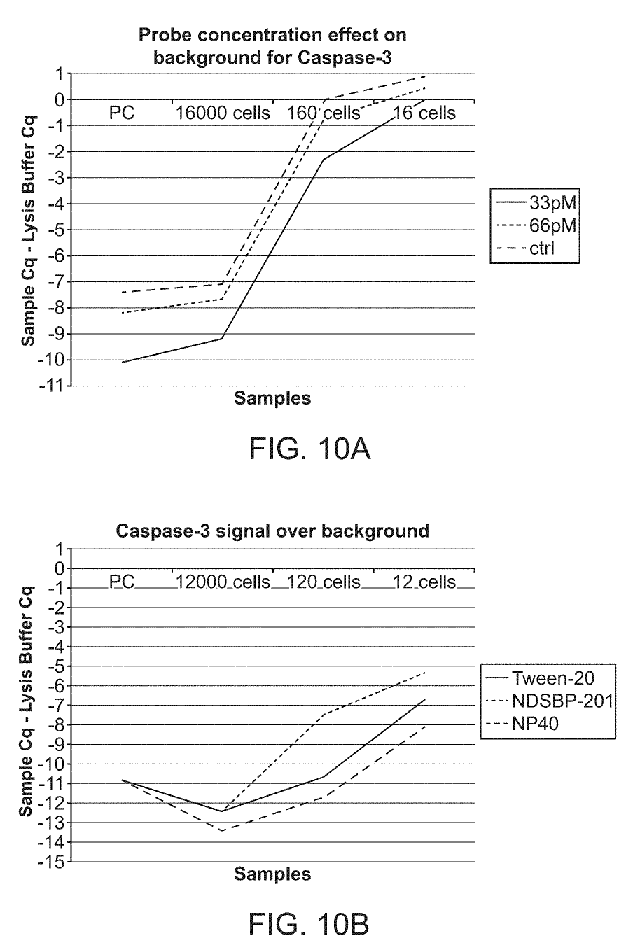

For example, if the proximity probes are diluted 4-fold prior to extension, the polymerase produces ¼×¼= 1/16 the amount of background casual signal (that is, background caused by random diffusion). After internal tests were performed using 1:5 and 1:10 dilutions of the extension template, roughly a 4 Cq unit difference was observed in background signals between the diluted extension templates and the original protocol, making it possible to detect EpCAM protein levels down to 16 cells in tubes (FIG. 9A-9B). When all the modifications described and justified above are added to the PEA protocol, the separation between protein signal from low cell input and background is much higher (FIG. 10A-10B).

### Example 2. Detection of Protein Expression in Single Cells by PEA Using the C₁™ Single-Cell Auto Prep System

Recent improvements in microfluidics and biochemistry have enabled single-cell molecular analysis, providing new insight into the heterogeneity of cell populations. The C₁™ Single-Cell Auto Prep System (Fluidigm) is an automated platform that streamlines the isolation and processing of 96 individual, live cells for RNA and DNA analysis. Single-cell protein profiling is a direct complement to genomic analysis as it provides additional insights into key molecular mechanisms and system biology. This example describes a highly multiplexed protein detection method (Proseek Multiplex Oncology I^{96×96}, Olink Bioscience) based on the Proximity Extension Assay technology (PEA) for use on the C₁™ Single-Cell Auto Prep System.

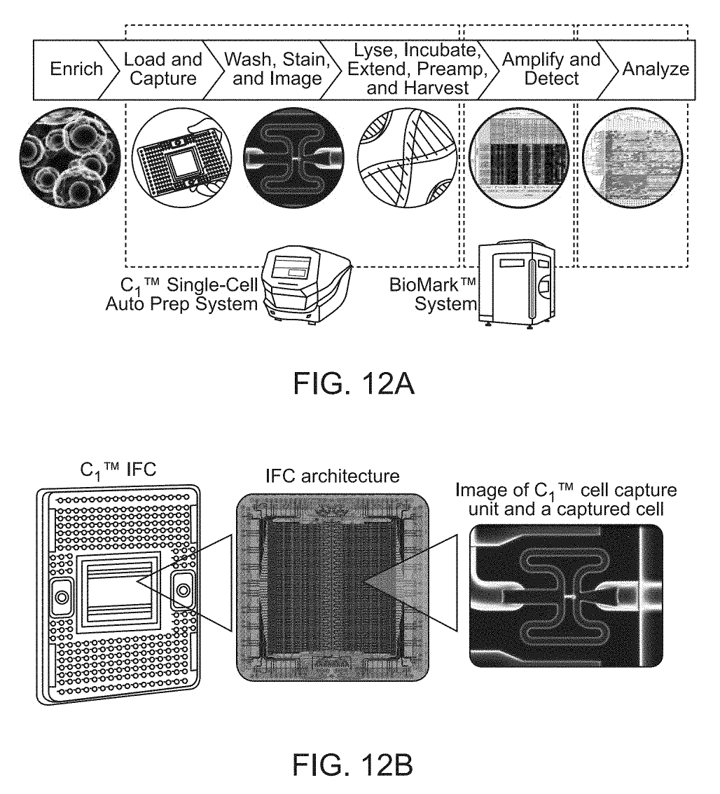

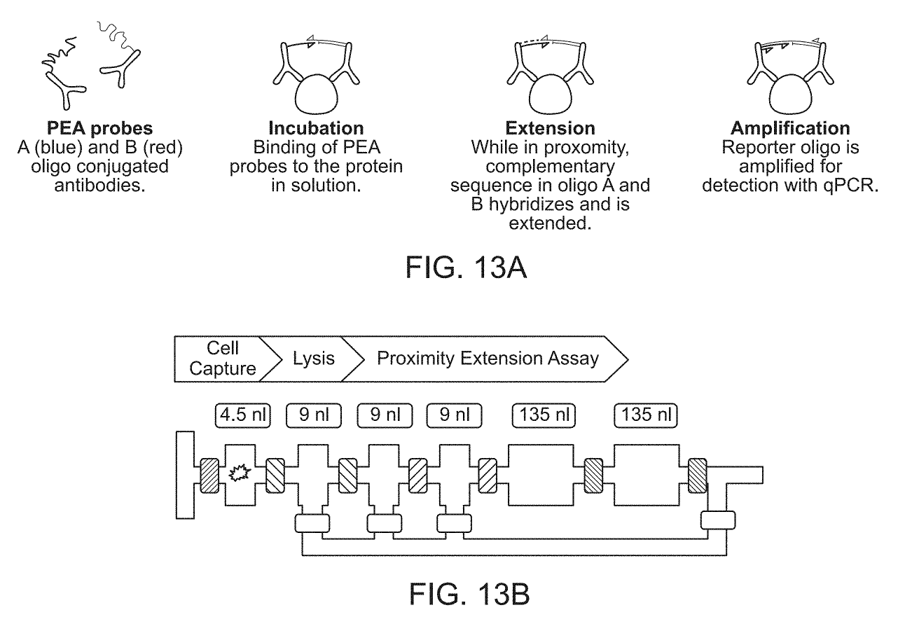

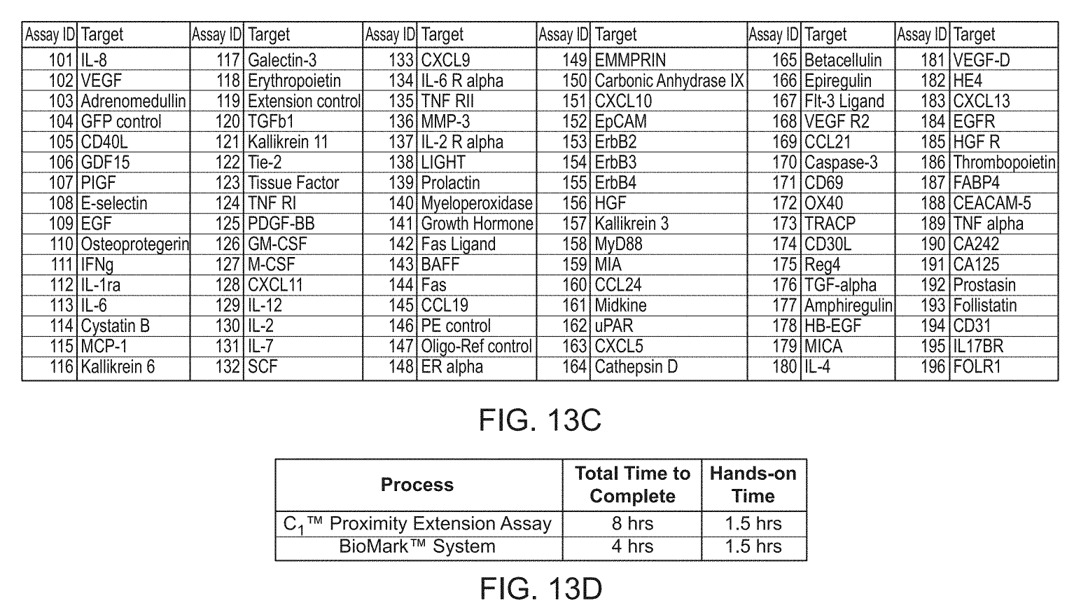

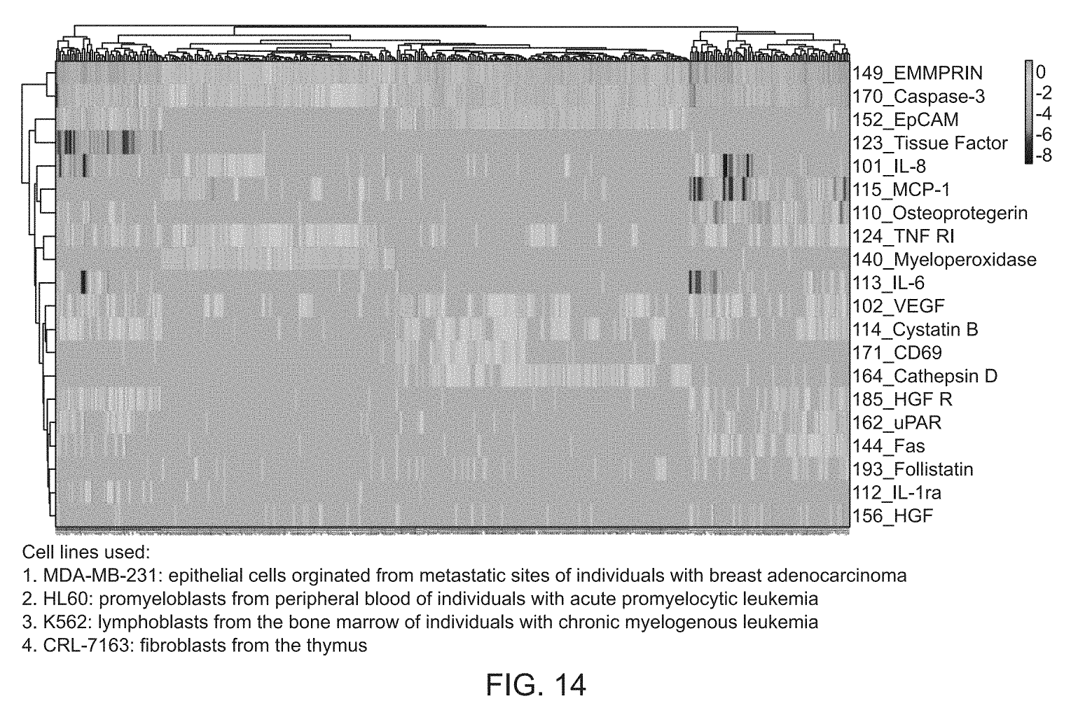

The C₁™ Single-Cell Auto Prep System is an integrated microfluidic system that provides a workflow for single-cell isolation, wash, live/dead cell staining, cell lysis, and further processing for molecular analysis from up to 96 cells per run (FIG. 12A-12B). This system was using with the Proximity Extension Assay technology (PEA) to develop a workflow for the automated analysis of the protein expression of single cells (FIG. 13A-13D). The method developed is based on the use of a PEA probe panel targeting 92 different proteins and of those, 66 correspond to intracellular proteins that can be detected in single cells (FIG. 13C).

The C₁™ Single-Cell Auto Prep System is composed of a controller instrument (FIG. 12A) and integrated fluidic circuits (IFC; FIG. 12B) containing 96 individual capture sites and dedicated nano-chambers for downstream reactions. The Fluidigm® integrated protein detection workflow allows for the simultaneous capture, lysis, incubation, extension, and amplification of reporter oligonucleotides from up to 96 cells using the C₁™ System.

In this system, each target-specific antibody was labeled with A or B oligonucleotides (PEA probes). During the incubation step, the PEA probes bind to the specific protein in the sample, bringing the A and B oligonucleotides closer in proximity. Hybridization of a complementary region within the A and B oligonucleotides takes place, followed by extension and amplification of the reporter oligonucleotide in a subsequent step, in presence of a DNA polymerase. Detection of the reporter oligonucleotide was performed by qPCR on a BioMark™ System (Fluidigm). Cycle threshold of the amplified reporter oligonucleotide reflects target protein abundance during the incubation step.

The C₁™ system includes a series of independent chambers and valves connected to the 4.5 nL single-cell capture site in a C₁™ Integrated Fluidic Circuit (IFC) (Fluidigm) (FIG. 13B). Each IFC contains 96 capture sites and each site has its own dedicated system of chambers, allowing all PEA steps to take place in a single run for 96 single cells in parallel.

An illustrative list of protein targets that can be analyzed is provided in FIG. 13C. In this example, the system has a single-cell to results turnaround time of 8 hours with 1.5 hours of hands-on time (FIG. 13D).

### Results

Results from PEA on plate-sorted cells were compared to results obtained from two independent C₁™ PEA experiments on single HL60 cells (FIG. 14). In general, results obtained from plate PEA on sorted cells confirmed results obtained by C₁™ PEA, with the exception of Tissue Factor. However, plate PEA signal for this specific target does not increase as expected when 10 and 50 cells are tested, suggesting that the high background signal of plate PEA could be affecting expression level results for this method.

A total of 401 single cells were analyzed (represented in columns in FIG. 14) in eight independent C₁™ PEA experiments for each of the four human cell lines MDA-MB-231 (n=54), CRL-7163 (n=83), HL60 (n=117), and K562 (n=147) (ATCC). Protein targets are represented in horizontal lines in FIG. 14. Across the two experiments performed for each cell line, 41, 31, 24, and 56 protein targets were detected as expressed in at least one single cell, respectively. Protein targets were considered expressed if ΔC_{T}=Sample C_{T}−(Avg. Background C_{T}−2*St. Dev. Background)<−0.4. FIG. 14 shows targets detected as expressed in a minimum of 10% of all single cells within each cell line analyzed. Of the 20 targets shown, seven exhibited somewhat specific expression levels in the following cell lines: Tissue Factor and IL-1ra in MDA-MB-231; Myeloperoxidase in HL60; CD69 and Cathepsin D in K562; MCP-1 and Osteoprotegerin in CRL-7163. Expression in specific cell lines and corresponding specific function were validated by literature analysis.

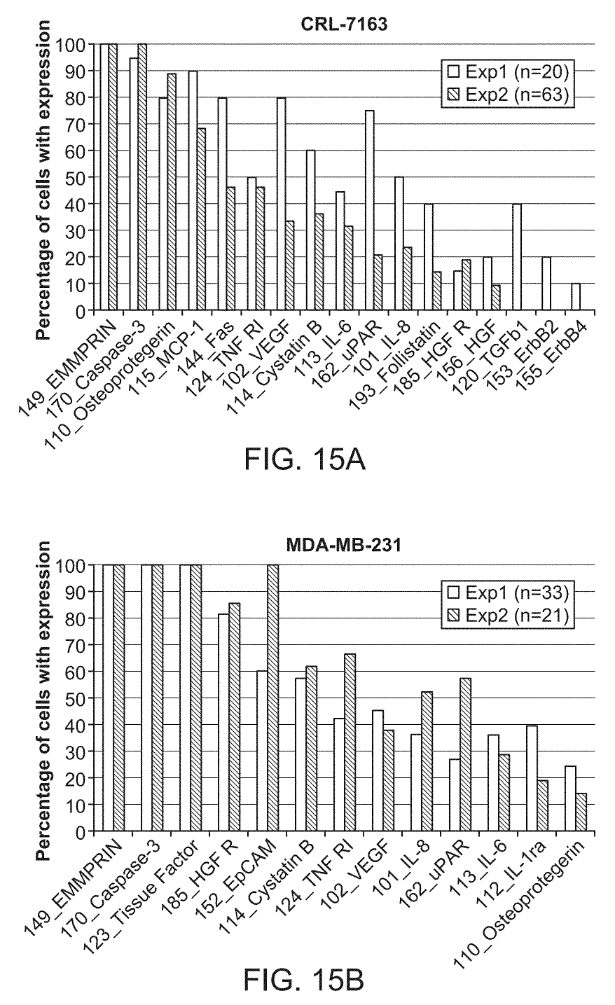

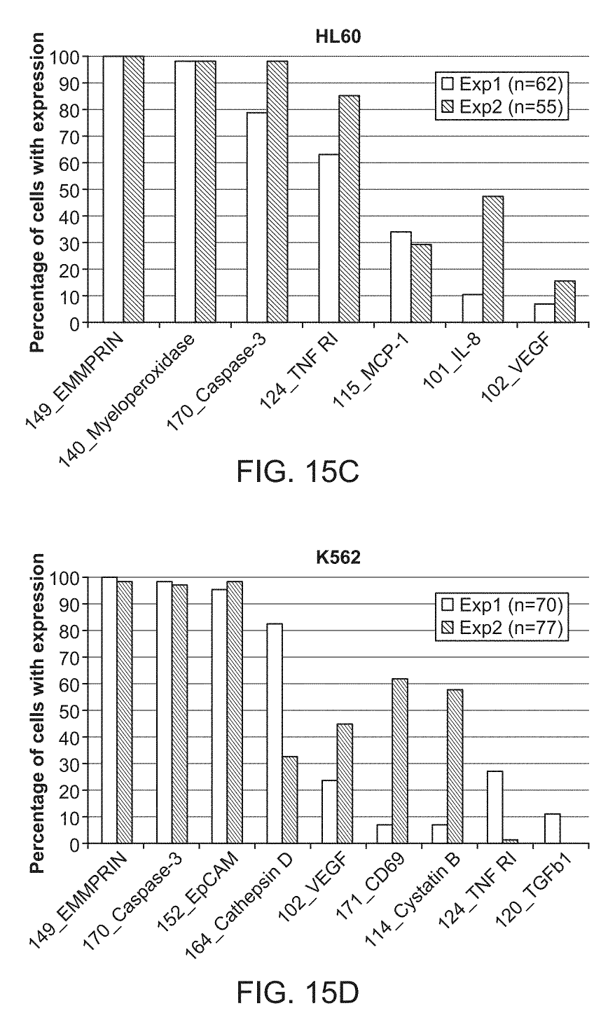

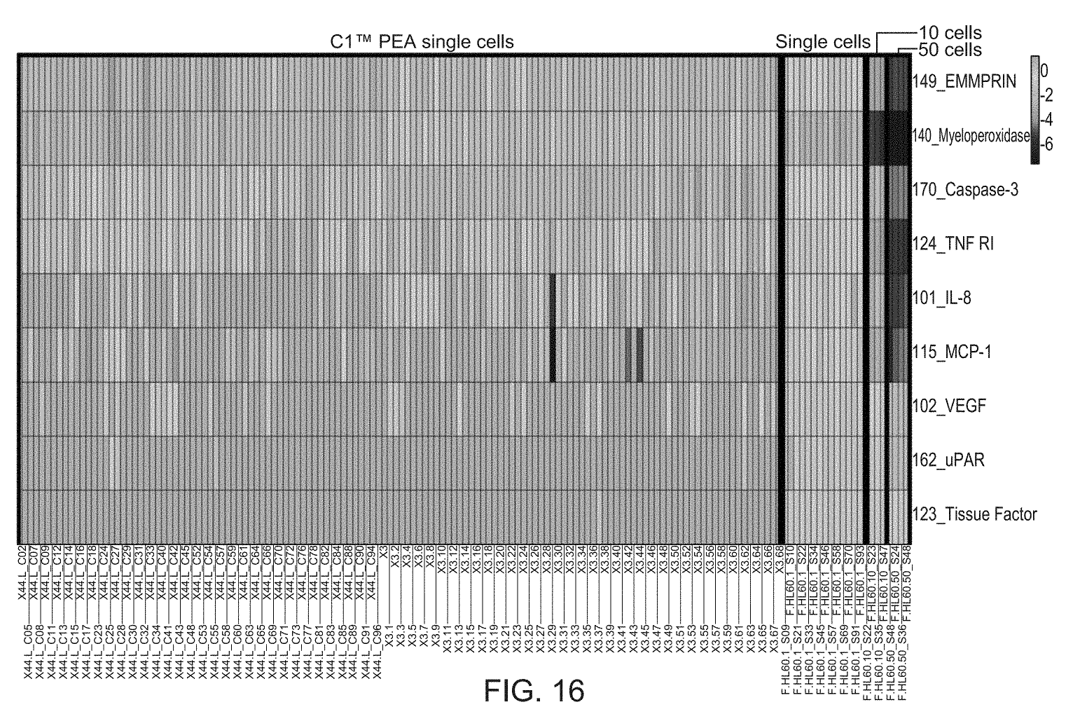

The results showed that most protein targets detected in the single cells were consistently detected across the experiments. FIG. 15 shows targets detected in specific cell lines tested across two independent C₁™ PEA experiments. As some level of variability of protein expression is typically observed at single-cell level, a more stringent criteria was used to select top targets expressed in the cell lines to evaluate experimental reproducibility: targets expressed in at least 10% of all single cells within at least one experiment with ΔC_{T}=Sample C_{T}−(Avg. Background C_{T}−2*St. Dev. Background)<−0.4 are shown. On average, 90% of the targets shown for each cell line were consistently expressed across the two experimental replicates at similar percentages of the cell population analyzed.

C₁™ PEA results for two specific targets (EpCAM and EMMPRIN) were validated on HL60 and K562 cells using orthogonal methods. In particular, EpCAM (low and high expression, respectively) and EMMPRIN (high expression in both cell types) antibodies conjugated with fluorescent dyes were used to evaluate expression levels of populations of cells with flow cytometry (Flow) and for on-chip immunofluorescence (IF) on single cells prior to C₁™ PEA. Flow and IF results were highly concordant with PEA results.

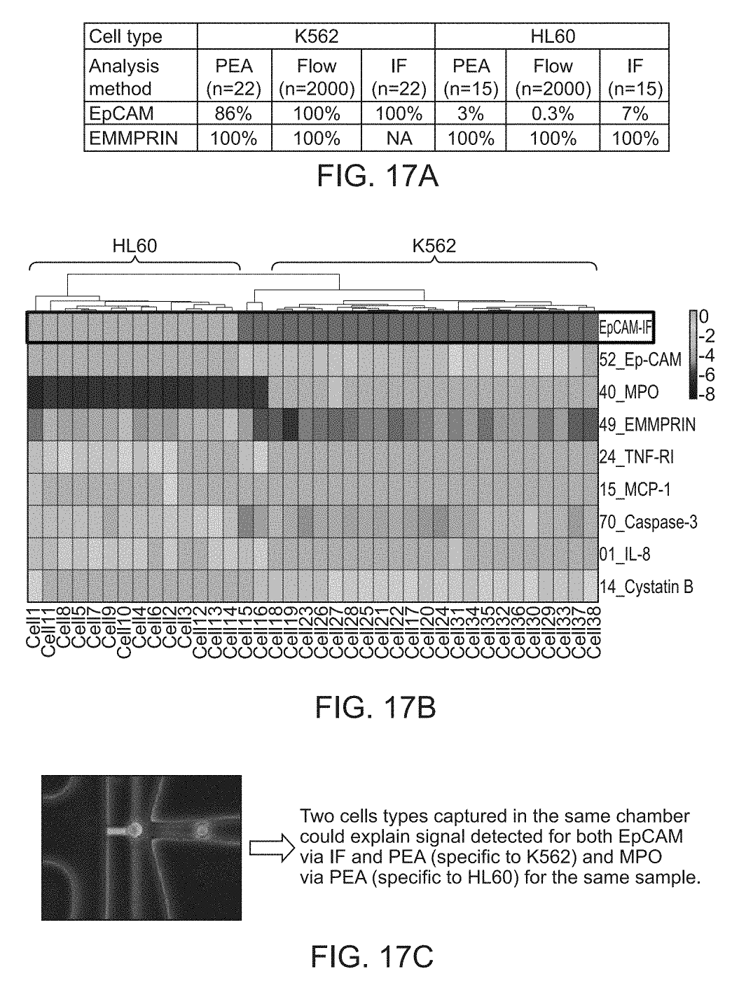

C₁™ PEA and on-chip immunofluorescence (IF) methods were performed to analyze the expression of protein targets, such as EpCAM, MPO, EMMPRIN, TNF-RI, MCP-1, Caspase 3, IL-8 and Cystatin B in single HL60 and K562 cells. As expected, K562 cells had high EpCAM expression confirmed by PEA and IF (FIGS. 17 A-B). Also, HL60 cells had high MPO expression levels confirmed by PEA. Two cells out of 38 analyzed with IF and PEA had results different than expected, presenting both EpCAM expression (IF and PEA) and MPO (PEA) (FIG. 17B). For one of those cells it was confirmed that two instead of one cell had been captured in the C₁™ IFC chamber (FIG. 17C).

### CONCLUSIONS

This example demonstrates automated protein detection from single cells using a C₁™ Single-Cell Auto Prep System single cell platform, with the ability to simultaneously process up to 96 single cells.

The method is sensitive enough to detect expression levels from single cells and can be used in combination with DNA and RNA profiling from single cells for further system biology studies. It is also consistent with other studies that target gene expression (Fang et al., BMC Cancer, 11:290 (2011); Van Lint et al., J Leuk Bio, 82(6):1375-1381 (2007; Yao et al., Int J Biol Scie 10(1):43-53 (2014); O'Donovan et al., Clin Cancer Res., 9:738 (2003); Doerfler et al., J Immunolo, 164(8):407-4079 (2000); Munz et al., Oncogene, 23(34):5748-58 (2004); Versteeg et al., Mol Med, 10(1-6):6-11 (2004); Murao et al., PNAS, 85(4):1232-1236 (1998); Hantschel et al., Mol Oncol 2(3):272-81 (2008); Lkhider et al., J Cell Science, 117(21):5155-5164 (2004); Burn et al., Blood, 84(8):2776-2783 (1994); Fisher et al., Cancer Research, 66:3620-3628 (2006).

The PEA probe panel from the Proseek Multiplex Oncology I^{96×96} kit, which targets 92 potential cancer-related targets, successfully profiled single cells derived from both cancer and normal tissue, grouping 98% of all cells analyzed (n=401).

### Materials and Methods

### Flow Cytometry

Flow cytometry was performed as follows. Separate 100 μL aliquots of 1×10⁶ of each of the two cell lines HL60 and K562 were washed with PBS and fixed with a final concentration of 4% formaldehyde. The cells were fixed for 10 minutes at 37° C. The tubes were then chilled on ice for 1 minute. The cells were then pelleted by centrifugation at 700 g for 5 minutes. The supernatant was aspirated and the cell pellet was re-suspended in 1.0 mL of 0.5% BSA in 1×PBS. Each of the two aliquots (one per cell line) was then divided into two samples and all four samples were washed by centrifugation at 700 g for 5 minutes. One sample from each cell line was re-suspended in 100 μL of EpCAM targeted antibody conjugated to AlexaFluor647 (Cell Signaling, Danvers, Mass.; 1:50 in 0.5% BSA in 1×PBS) and one sample from each cell line was re-suspended in 100 μL of CD147 (EMMPRIN) targeted antibody conjugated to AlexaFluor488 (BioLegend, San Diego, Calif.; 1:50 in 0.5% BSA in 1×PBS). All four re-suspended samples were incubated in their respective antibodies for 1 hour at room temperature in the dark. Cells were then washed in 1.0 mL of 0.5% BSA in 1×PBS by centrifugation at 700 g for 5 minutes. Each sample was re-suspended in 0.5 mL of 1×PBS. Flow cytometry was performed on an FACSARIA III instrument (Becton Dickenson).

### C₁™-PEA

The C1™ IFC was primed using standard protocols (see, e.g., the User Guide titled “C₁™ System for DELTAgene Assays” (Fluidigm Document ID 100-490″), available from Fluidigm.

A cell suspension of a pre-determined concentration (e.g., 60,000-70,000/mL) in native medium was made prior to mixing with a suspension reagent (C1™ Single-Cell Auto Prep Module 1 Kit, Fluidigm PN 100-5518) and loading onto the C1™ IFC. The cells were combined with the C₁™ Cell Suspension Reagent at a ratio of 3:2 and 5-20 μl of the final cell mix was loaded onto the C₁™ IFC through the “cell loading” inlet.

### Immunofluorescence on C₁™-IFC

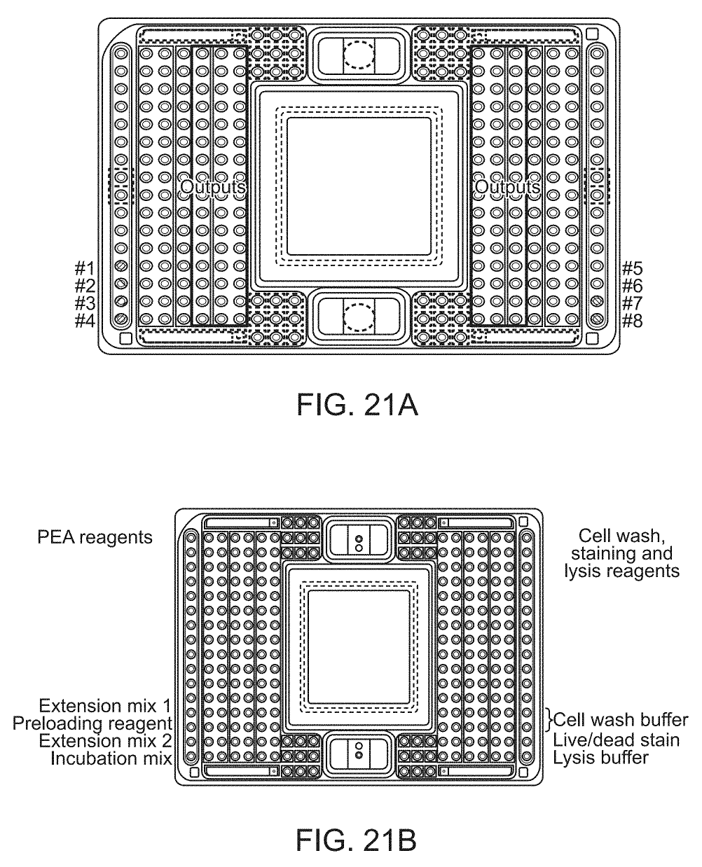

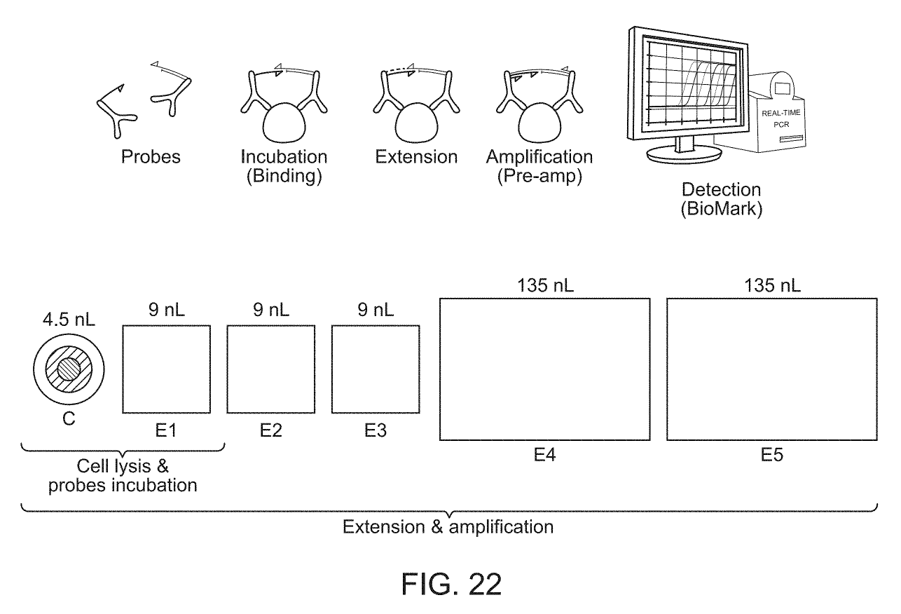

Fluorescently labelled antibodies were prepared in the recommended concentration for standard immunofluorescence in cell wash buffer (C1™ Single-Cell Auto Prep Module 1 Kit, Fluidigm) spiked with 0.5% bovine serum albumin (BSA) solution. The antibody mix was pipetted into C1™ IFC reagent inlet #7 (inlet numbering shown in FIG. 21). The cells were introduced into the capture site, washed with cell wash buffer, incubated with the antibody mix in the capture site for 20 minutes at room temperature, and then washed. The cells were then imaged on a fluorescent microscope compatible with C1™ IFCs.

### Protein Expression by C₁™-PEA—Amplification

After immunofluorescence analysis, the cells were analyzed in a PEA reaction. Briefly, the C1™ IFC was placed into the C1™ Single-Cell Auto Prep System. The cell lysis mix was loaded into the first reaction chamber (9 nL) and incubated at room temperature for five minutes. The incubation mix containing the PEA probes was then loaded into the second and third reaction chambers (9 nL+9 nL) and incubated for 37° C. for one hour. Extension mix 1 was then loaded into chamber four (135 nL) and extension mix 2 into chamber five (135 nL) and the standard Olink Bioscience thermal protocol for extension and amplification was performed (50° C. for 20 minutes, 95° C. for 5 minutes, then 17 cycles of 95° C. 30 seconds, 54° C. for 1 minute, and 60° C. for 1 minute). PEA product was harvested up to 16 hours after the last PEA thermal step was completed. The harvested PEA product was then pipetted into a new 96-well plate for further analysis.

### Protein Expression by C₁™-PEA—Detection

The C1™-PEA product and in-tube controls (see below) were detected using an Olink Bioscience standard detection protocol with a Fluidigm 96.96 GE IFC. In this example, 1.4 μL of harvest PEA product or in-tube control PEA was added to 3.6 μL of detection mix.

The Fluidigm 96.96 GE IFC was primed and loaded with 4 μL of each reaction and 4 μL of each assay from the 96-well assay plate provided in the Olink Bioscience PEA Mulitplex Detection Kit. The RT-PCR was run using the Olink Bioscience Protein Expression 96×96 Program on the Fluidigm BioMark™ system. The reaction included an initial thermal mix (50° C. for 2 minutes, 70° C. for 30 minutes, and 25° C. for 10 minutes) followed by a hot start (95° C. for 5 minutes) and PCR cycles (40×95° C. for 15 seconds and 60° C. for 1 minute).

### Reagents

The lysis mix contained 27 μL of C1™ Lysis Plus Reagent (C1™ Single-Cell Auto Prep Module 2 Kit, Fluidigm, PN 1000-5519) and 3 μL of cell wash buffer (Fluidigm) of which 10 μL was pipetted into inlet #8 (inlet numbering shown in FIG. 21). The final concentration of detergent in the lysis buffer was above 1.0%.

The C1-PEA incubation mix consisted of 14.69 μL of Incubation Solution (Olink Bioscience), 2.5 μL of Incubation Stabilizer (Olink Bioscience), 3.28 μL of A-Probes (Olink Bioscience), 3.28 μL of B-Probes (Olink Bioscience), and 1.25 μL of C1™ Loading Reagent (C1™ Single-Cell Auto Prep Module 2 Kit, Fluidigm, PN 1000-5519) of which 10 μL was added to inlet #4.

The Extension Mix 1 was composed of 27.9 μL PEA Solution (Olink Bioscience), 6.3 μL of C1™ Loading Reagent (Fluidigm), and 90.8 μL high purity PCR-grade water of which 25 μL was added to inlet #1.

The Extension Mix 2 was composed of 1.4 μL PEA Enzyme (Olink Bioscience), 0.6 μL of PCR Polymerase (Olink Bioscience), 6.3 μL of C1™ Loading Reagent (Fluidigm), and 116.7 μL of high purity PCR-grade water of which 25 μL was pipetted into inlet #3 (FIG. 21 inlet numbering). Harvest Solution (Fluidigm) is added to all four reservoirs of the C₁™ IFC at 150 μL.

The detection solution was prepared by adding 268 μL of Detection Solution (Olink Bioscience), 3.86 μL of Detection Enzyme (Olink Bioscience), 1.54 μL of PCR Polymerase (Olink Bioscience), and 112.6 μL of high purity PCR-grade water.

Preparing in-Tube Controls

At least two in-tube controls were performed alongside the IFC, a no-protein control (NPC) and positive protein control (PPC). These controls were conducted with either 1 μL of Cell Wash Buffer (NPC) or 1 μL of cell lysate (PPC; cells lysed with the lysis mix as prepared above incubated for 5 minutes at room temperature) and 1.33 μL of the incubation mix. This reaction was incubated for 15 minutes at 25° C. and then for one hour at 37° C. After incubation, 10 μL of Extension Mix 1 and 10 μL of Extension Mix 2 were added to the incubated in-tube controls. The thermal protocol for the IFC was used.

### Example 3. Additional Analyses—PEA Using C1™ System

This example additionally illustrates single cell protein analysis parameters using a Fluidigm C1™ single cell detection system.

For single cell analysis, PEA occurs in four steps: lysis of the cell, incubation with PEA probes, extension, and PCR amplification. Typically, lysis of the cell is performed in a non-ionic detergent to maintain the native structure of the proteins. In this example for an illustrative protocol, cells captured on the C1™ Integrated Fluidic Circuit (IFC) were lysed with C1™ Lysis Plus Reagent (Fluidigm) in a final solution that contained 1.5% NP-40, 2% Prionex® gelatin, 2 mM TRIS HCl pH 8.0, 10 mMKCl, 0.1% v/v Tween 20, and 40% v/v HBSS.

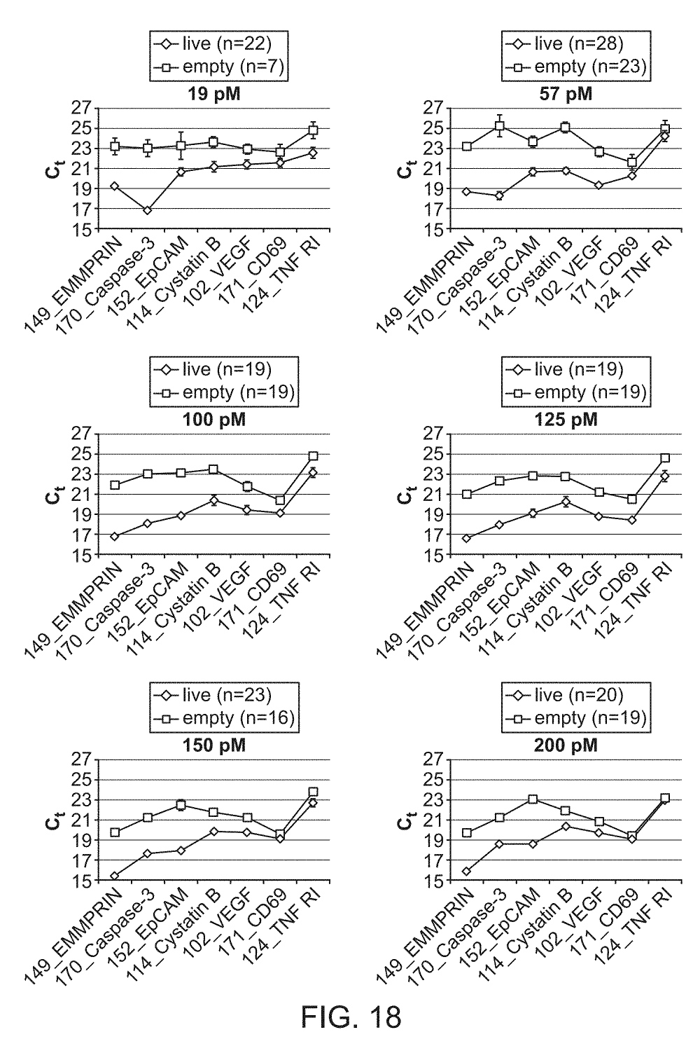

Volume-to-volume ratios of PEA reagents obtained from 0-Link Biosciences for incubation and extension/amplification steps were altered to enhance performance for single cell protein analysis. Ratios and calculated probe amounts were tested in the range of 19 pM to 200 pM. FIG. 18 shows the results for a subset of targets for PEA with probe concentrations that vary between 19-200 pM in the incubation reaction. The best separation in this analysis between the average Ct for live cells and background occurred at concentrations of 100 and 125 pM.

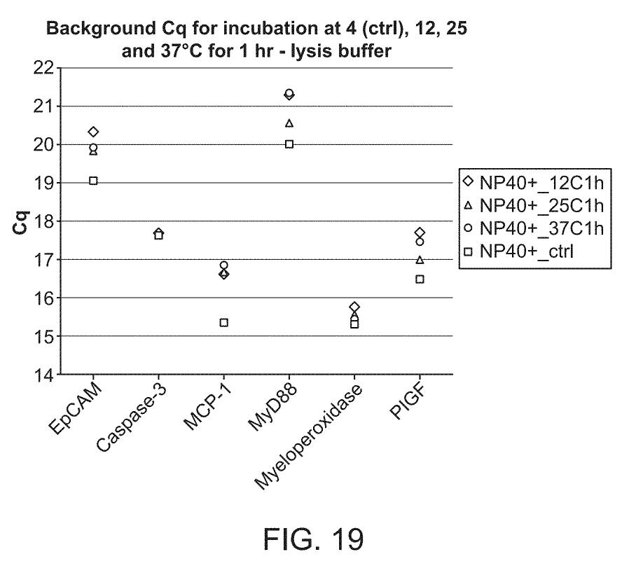

The incubation period employed was 1 hour at 37° C., as initial experiments had shown that shorter incubation times at higher temperatures decreased background signal relative to longer incubation times at lower temperatures, such as overnight at 4° C. (FIG. 19).

A comparison of protocols separating the extension/amplification reaction components into two parts, and combining the extension and amplification reaction components was performed. This experiment thus evaluated separating the polymerase enzymes for the PEA and amplification reactions from the probe hybridization solution until mixing just prior to initiation of the extension/amplification thermal protocol. Single cells (96) in parallel were evaluated and all reagent mixes were prepared and added to the chip. The reagent mixes were present in the chip for up to 3 hours until the reagents were delivered to the reaction chambers. Preliminary experiments conducted using tube incubations indicated that pre-incubating either the PEA enzyme or PCR polymerase with a PEA solution provided in a PEA reagent kit from 0-link Biosciences resulted in overall lower signal production than freshly prepared PEA mix (Table 1, columns “Test 2” and “Test 3”). Incubation of the PEA solution with both PEA enzyme and PCR polymerase resulted in the poorest signal production (Table 1, column “Test 4”, likely due to the exonuclease activity of both enzymes acting on the PCR primers included in the PEA solution. In view of these results, the protocol for C₁™-PEA employed in the illustrative protocol below uses one inlet for the PEA solution and a separate inlet for a mix containing the PEA enzyme and PCR polymerase to reduce the period of time in which the PCR primers in the PEA are in contact with the polymerases.

Table 1: For each test 1-4, mix 1 and mix 2 were incubated for 1 hour and 20 minutes at 30° C. in PCR tubes on a standard thermal cycler. The time of incubation was determined from the amount of time the extension/amplification reagents would be sitting on the C1™ IFC prior to loading into the reaction chamber, i.e. the lysis (5 minutes)+incubation (1 hour)+loading (15 minutes) times. Heat from the thermal chuck is not restricted to the PDMS component of the IFC, i.e. the carrier will be heated as well, it was estimated that the temperature in the reagent well during the 37° C. incubation would be approximately 30° C. Thus, the incubation temperature for mix 1 and 2 (tests 1-4) was 30° C. A recombinant protein pool was used as the PEA sample. A reference sample of freshly prepared extension/amplification mix which was added to the incubated sample just prior to beginning the thermal protocol was also prepared. Each scenario, including the “fresh” reference sample, was run in duplicate and analyzed on a BioMark instrument with a standard Olink PEA detection reagents and protocol. The ΔCts were calculated by subtracting the average Ct of all 96 assay results across both replicates for the “fresh” sample from each of test 1-4, respectively. In the table: Enz., PEA Enzyme; Pol, PCR Polymerase; Soln, PEA Solution; avg, average; sd, standard deviation; max, maximum; min, minimum.

TABLE 1
ΔCₜs (Avg of Fresh - Test)

| | Test 1 | Test 2 | Test 3 | Test 4 |
|---|---|---|---|---|
| Mix 1 | Enz. + Pol | Enz. | Pol | All |
| Mix 2 | Soln | Pol + Soln | Enz. + Soln | |
| avg | 0.980 | −0.853 | −1.001 | −5.574 |
| sd | 0.809 | 0.820 | 0.963 | 0.835 |
| max | 3.740 | 2.543 | 2.271 | −2.615 |
| min | −0.591 | −2.718 | −5.493 | −7.679 |

Two factors were additionally considered in this example in the logistics of the single cell analysis. Reagents are loaded into the reaction chambers at 25° C. The time for loading the reagents depends on the size of the reaction chambers used for that particular step. The loading times are as follows: lysis solution, 30 seconds (9 nL chamber), incubation solution, total of 1 minute (two 9 nL chambers), the first of the two extension/amplification solutions, 15 minutes (135 nL chamber), and the second of the two extension/amplification solutions, 15 minutes (135 nL chamber). After the initial lysis solution is added, a mixing step is performed on the C₁™ IFC at 25° C. as additional reagents are added. Mixing occurred after the reagents are delivered to the specific chambers, and before the incubation and thermal protocols. After the incubation solution is loaded there is a 15 minute mixing time and after both extension mixes were loaded there is a 25 minute mixing time.

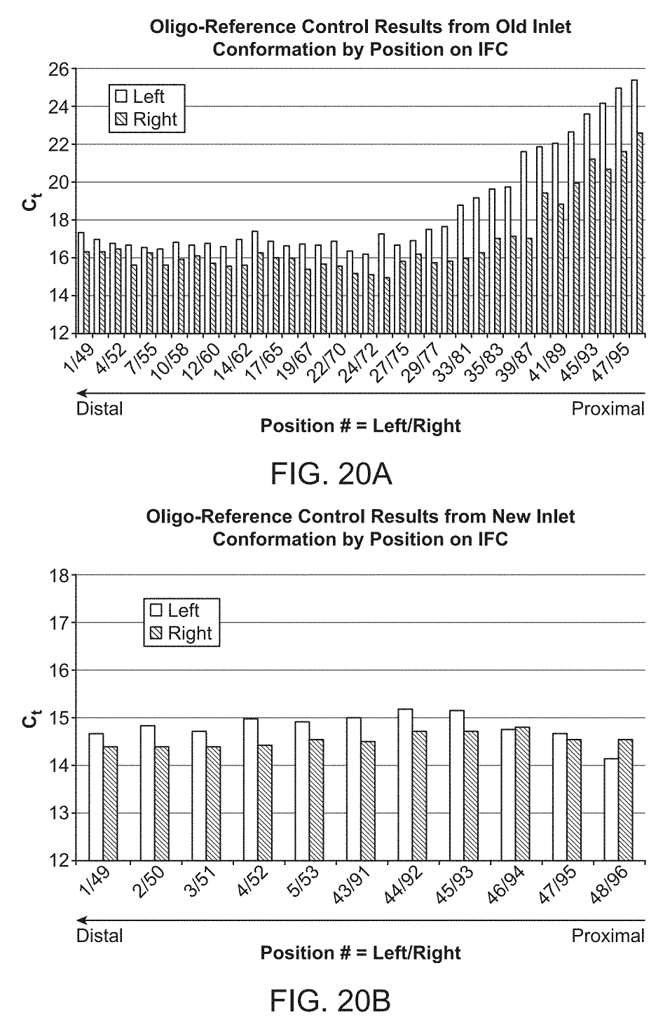

Protocols using the Fluidigm C1™ single cell analysis system typically involve introduction of reagents for enzymatic reactions from two inlets, in this example, inlets #7 and 8 (inlet numbering shown in FIG. 21), using a multiplexer structure. This structure is shared by all reagents pipetted in inlets #5, 6, 7 and 8 for delivery to the chips reaction chambers. In this experiment reagents introduced into inlets #5 and 6 corresponded to cell wash buffer (1×HBSS), which is high in salts. Early iterations of the C₁™-PEA method introduced the full PEA reagent mixture for the extension and amplification steps (i.e. PEA solution, PEA enzyme, and PCR polymerase) from inlets #7 and 8. However, non-uniform results were observed from the PEA controls across the 96 reaction positions of the C₁™ IFC, even though all positions on the chip should provide similar signals for the controls. FIG. 20A shows that the Ct values for the PEA controls were highest at the positions most proximal to the entry point of the reagents (i.e., positions 48 and 96) and were progressively lower towards the most distal positions (i.e. positions 1 and 49). This may be due to residual high salt buffer left behind in the multiplexer shared by the PEA extension and amplification reagents, which could be detrimental to the PCR in reaction chambers closest to that structure, that is, 48 and 96. To evaluate this, the inlet positions for the PEA reagents for extension and amplification steps were switched with other reagents that are not sensitive to high salt concentrations (in this case, the cell viability stain and lysis mix). FIG. 20B provides data that confirmed that the positionally-related performance was abrogated by the switch. FIG. 21 shows the final configuration of reagents loaded into the C1 TM chip carrier.

Additional experiments demonstrated that lysis with 0.5% NP-40 provided sensitive detection, but nuclear compartment largely intact.

In a further experiment, combining cell lysis and probes incubation steps together at chambers 0-1 highly improved PEA performance (see, FIG. 21).

### Further Evaluation of the C1™ Single Cell PEA System

Increasing sensitivity of C₁™-PEA to detect a greater number of expressed targets at single-cell antigen levels is desirable. Various parameters are additionally evaluated:

The extension/PCR amplification may also be performed on a secondary Access Array IFC. This provides for a greater ratio of extension/PCR amplification reaction volume to incubation reaction volume compared to that in the examples above using a C1™ IFC alone. In this example, an IFC is typically used that permits loading the harvest from most samples of two C₁™-PEA IFCs and has space to load positive and negative controls. Having all C1™ IFC chambers available for incubation will provide a 1:3 ratio of sample volume to incubation mix volume. Specifically, chambers 0-3 will be used for lysis (30.5 nL total) and a diluted (relative to the manufacturer's recommendation) mix of incubation reagents will be introduced into chamber 4 (chamber 4 is 135 nL) such that 124 nL of mix introduced into chamber 4 is the incubation reagents and 11 nL is water so that of the total (165.5 nL), one-third of the volume is represented by incubation mix. The incubated material will then be harvested, which will result in 165.5 nL of sample to 4 μL of harvest volume (i.e. 0.041× of volume is incubated sample).

As the harvest volume can be variable and as low as 3 μL, a volume of harvest is used that can be consistently obtained for every sample to use in the reaction preparation for the secondary IFC, i.e. 2 μL. As 4 μL of sample mix is needed for loading, half of the sample volume is from harvest material, thereby achieving a ratio of incubation reaction to extension/amplification reaction volume that is equal to 0.02. For PCR inefficiencies that are a result of inhibition by components in the incubation mix, this lowered relative volume of incubation reagents improves the PCR efficiency. The volumes are adjusted for the secondary IFC. For example, for an AA192.24 Fluidigm IFC, the volume for loading an assay well is 4 μL. Thus, the assay mix is represented by 0.21 μL of PEA enzyme, 0.084 μL of PCR polymerase, 1× Access Array loading reagent, and PCR-grade water. The AA192.24 IFC is loaded on an AXHT controller and cycled on an FC1 cycler using the Std-PEA extension/amplification thermal protocol. Samples are harvested and analyzed using the standard PEA detection protocol.

It is understood that the examples and embodiments described herein are for illustrative purposes only and that various modifications or changes in light thereof will be suggested to persons skilled in the art and are to be included within the spirit and purview of this application and scope of the appended claims.

In addition, all other publications, patents, and patent applications cited herein are hereby incorporated by reference in their entirety for all purposes.

1. A method of detecting a target analyte in a single cell, the method comprising:
a) isolating the single cell;
b) incubating the single cell in a lysing buffer comprising a detergent present at a concentration below the critical micelle concentration to obtain a cell lysate;
c) incubating the cell lysate with two or more proximity extension probes in a binding reaction at an incubation temperature from about 15° C. to about 50° C. for a length of time from about 5 minutes to about 6 hours under conditions where the proximity extension probes bind to the target analyte, if present, in the cell lysate;
d) incubating the binding reaction with an extension mix that comprises a polymerase, wherein hybridized oligonucleotide components of the proximity extension probe are extended by the polymerase to produce extension products;
e) detecting the extension products.

2. The method of claim 1, wherein at least one of the proximity extension probes comprises an antibody as an analyte binding component.

3. The method of claim 2, wherein the concentration of the proximity probe in the binding reaction ranges from about 1 pM to about 1 nM, or from about 10 pM to about 100 pM, or from about 20 pM to about 200 nM.

4. The method of claim 1, wherein steps (a)-(e) are performed in a microfluidic device.

5. A method of detecting a target analyte in a single cell, the method comprising:
a) isolating the single cell, wherein isolating the single cell comprises isolation of individual cells into droplets in a microfluidic device;
b) incubating the single cell in a lysing buffer comprising a detergent present at a concentration below the critical micelle concentration to obtain a cell lysate;
c) incubating the cell lysate with two or more proximity extension probes in a binding reaction at an incubation temperature from about 15° C. to about 50° C. for a length of time from about 5 minutes to about 6 hours under conditions where the proximity extension probes bind to the target analyte, if present, in the cell lysate;
d) incubating the binding reaction with an extension mix that comprises a polymerase, wherein hybridized oligonucleotide components of the proximity extension probe are extended by the polymerase to produce extension products;
e) detecting the extension products.

6. A proximity extension detection probe set for detecting interaction of a protein with a single-stranded nucleic acid, wherein the probe set comprises a first proximity probe that comprises a binding region that binds to the protein and a first oligonucleotide comprising an interacting region; and a second proximity probe that comprises an oligonucleotide that comprises a segment that hybridizes to the single stranded nucleic acid and a segment that comprises an interacting region that is complementary to the interacting region of the first proximity probe, wherein, when the protein is bound to the single-stranded nucleic acid, the interacting region of the first probe hybridizes to the complementary segment of the second probe.
